(19) United States
(12) Patent Application Publication
(10) Pub. No.: US 2005/0095351 A1
(43) Pub. Date: May 5, 2005

(54) Method, apparatus and system for nanovibration coating and biofilm prevention associated with medical devices

(76) Inventors: Jona Zumeris, Nesher (IL); Zadick Hazan, Zichron Yakov (IL); Yanina Zumeris, Nesher (IL)

Correspondence Address: Bernard Malina, Esq.;Malina & Associates, P.C., Suite 501, 60 East 42nd Street, New York, NY, 10165, US

(21) Appl. No.: 10/986,530

(22) Filed: Nov. 10, 2004

Related U.S. Application Data
(60) Provisional application No. 60/556,266, filed on Mar. 24, 2004.

(30) Foreign Application Priority Data
May 29, 2003 (WO) PCT/IL03/00452

(51) Int. Cl. A61L 2/00

(52) U.S. Cl. 427/2.1

(57) ABSTRACT

An acoustic indwelling medical device system, which may include a vibration apparatus and at least one transducer, may be integrated with standard medical devices. This acoustic system may use electric signals to enable the transducer to generate nanovibrations within the indwelling medical device system, to inhibit the entry of microorganisms from external sources. Such vibrations may enable dispersal of microbe colonies, thereby preventing or dispersing biofilm that may cause infections.

### RELATED APPLICATIONS

This application claims the benefit of PCT Patent Application No. PCT/IL03/00452. filed May 28, 2003, U.S. patent application Ser. No. 10/445,956, filed May 28, 2003, and Provisional U.S. Patent Application No. 60/556,266, filed Mar. 24, 2004, which are incorporated in their entirety herein by reference.

### FIELD OF THE INVENTION

The present invention relates generally to the fields of invasive medical devices, medical device associated infections or indwelling medical devices and more specifically, to a method and system for preventing or treating biofilm or bacteria or micro organisms associated with such devices.

### BACKGROUND OF THE INVENTION

Invasive devices or indwelling medical devices such as medical device associated infections, e.g., intravascular devices, non-needle connectors, endothracheal ventilation tubes, intrauterine devices, central venous catheters, drug delivery tubing and parts of the tubing mechanically connecting with electro-mechanical devices (e.g., peristaltic pumps), mechanical heart valves, pacemakers, peritoneal dialysis catherers, tympanostomy tubes, prosthetic joints, voice prostheses, urinary catheters, porta-caths, etc. (hereinafter referred collectively to as “medical devices”), which are passed directly or indirectly through body orifices, vessels, or through an opening made in a patient's skin, are associated with a significant risk of infection (and other related medical problems). All device associated infections are due to biofilm formations on foreign material introduced into the body, or to the absorption of protein or minerals (accretions) leading to clots that build on these foreign materials. For example, infections are associated with the development of pathogenic microorganisms in the form of biofilm on inner or outer surfaces of the medical devices and/or between the tissue surface and the foreign material introduced.

Life-threatening systemic infections may occur as a result of such biofilm formations. Therefore, the medical device has to be removed. The patient often requires antibiotic treatment and re-insertion of new medical devices. This leads to further risk of infection, as well as significant unpleasantness and expense.

Known methods for treating and/or preventing catheter-associated infections include the insertion of catheters using aseptic techniques, the maintenance of the catheter using closed drainage, the use of special non-standard medical devices, and the use of anti-infective agents.

Currently available solutions often involve antibiotic or disinfectant coating of the medical device. These solutions are not satisfactory and expensive and sometimes are even implicated with aggravation of the problems they were intended to solve. The potential of antibiotic resistance to a coating is an additional negative consideration, since biofilms can provide the conditions for bacterial resistance to develop.

Known methods of preventing bacterial biofilm formation on water-filled tubes include using axially propagated ultrasound and methods using low frequency ultrasound of high power density combined with aminoglycoside antibiotics for killing biofilms. The usage of ultrasound for transmitting of mechanical vibrations is associated with many technical difficulties associated with materials properties, their technical parameters and configuration. In many cases, materials of medical devices have elastic features such as those constructed with plastics and rubber latex. Their inner and outer surfaces have complicated configurations (for example, many have conusoidic shape). These conditions result in the non-homogeneous distribution of mechanical vibrations (ultrasound). Such vibrations in many cases can cause high temperatures and these negative and uncontrolled phenomena can affect human cells. These are the main obstacles in applying such vibrations for continuous biofilm prevention. It is known that biofilm formation begins after several hours and can be continuous for several days. Another obstacle appears while using ultrasound-elastic mechanical vibration waves transmitted to the external and internal surfaces of indwelling medical devices. These are the so-called “dead points” in which the amplitude of vibrations is minimal and equal to zero. “Dead points” are the places where conglomeration of bacteria colonies form, and biofilm formation process begins.

A device that would markedly decrease the need for repetitive medical devices replacement, and allow for a significant decrease in the associated morbidity, would be advantageous.

### SUMMARY OF THE INVENTION

There is provided, in accordance with the embodiments of the present invention, an apparatus, system, and method for using nanovibration coating for preventing or treating pathogenic microorganisms (infections) associated with medical devices. When single bacteria (planktonic, free floating) attach to a solid surface, it establishes a contact with it and locks on it. Within a few hours, a carpet of bacteria develops and spreads along the tissue and device. These bacteria soon start to secrete a polysaccharide substance called glycocalyx in which bacteria take shelter and thus become greatly resistant to disinfection and to the body immune system. The ability to prevent these biofilm formations would constitute a great step forward in prevention of device associated infection (resulting from biofilm formation on the device).

According to some embodiments of the present invention, by means of applying combinations of mechanical vibrations and various techniques for their propagation, we create on internal, external and torsion surfaces of medical devices nanovibrations of very small amplitude and pressure. This antibacterial coating is herein known as a “nanovibration coating” (shield). The magnitude of nanovibrations is several/or ten times smaller in comparison to the size of bacteria and such small vibrations do not increase temperature. It is possible to control magnitude, direction, and rate of nanovibrations on external and internal surfaces of a medical device. It is possible to create at the same time propagation of elastic waves of different types (different harmonics and directions). This creates spacious nano elastic waves on internal, external and torsion surfaces of a medical device. The range of waves in these spacious fields is smaller by several times in comparison with bacteria size, and “dead points” (with zero amplitude) are avoided.

According to some embodiments of the present invention, an acoustic system, which may include apparatus with nanovibration coating effect and at least one transducer, may be integrated or attached to standard medical devices. This nanovibration coating system may use electric signals to enable the nanovibration coating transducer (external and or internal and or torsion surfaces) to generate nano range coating vibrations on the medical devices (external and or internal and or torsion surfaces), to inhibit the entry of microorganisms or prevent biofilms formation on external or internal or torsion surfaces. Such nanovibration coating enables dispersal of microbe colonies, thereby preventing or dispersing biofilm, that may cause infections. For example, mechanical nanovibration coating such as nano micro vibration may be generated by the transducer elements, such as elements with piezoelectric effect and/or piezomagnetic effect. Nanovibration transducers convert harmonic or impulse electrical energy by means of elastic mechanical nano waves of one or multi degrees of freedom. Generation of nanovibration waves (of longitudinal or flexural torsion or multi degree of freedom) on the medical devices surfaces (nanovibration coating) can have traveling or standing wave forms. The result is a marked decrease in the amount of biofilms which are the source of infections.

A nanovibration coating processor may include a power supply, controller, one or more oscillators and a switching device. The strength, duration, type, location, etc. of the nanovibration coating waves may be controlled by the vibration processor and its components.

### BRIEF DESCRIPTION OF THE DRAWINGS

The principles and operation of the system, apparatus, and method according to the present invention may be better understood with reference to the drawings, and the following description, it being understood that these drawings are given for illustrative purposes only and are not meant to be limiting, wherein:

FIG. 1 is a schematic illustration of an acoustic medical device system and the main parts of vibration device with controller, for nanovibration coating of the surfaces for biofilm formation prevention and/or dispersing biofilm formations, according to some embodiments of the present invention;

FIG. 2 is a schematic illustration of elastic acoustic longitudinal waves of various modes in a medical device system for nanovibration coating of the surfaces for preventing biofilm formation and/or dispersing biofilm formations, according to some embodiments of the present invention;

FIG. 3 is a schematic illustration of a spectrum plot of a nanovibration coating in the medical device exhibiting longitudinal, bending, torsion modes and combinations thereof for preventing biofilm formation and/or dispersing biofilm formations, according to some embodiments of the present invention;

FIG. 4 is a schematic illustration of a switching device having at least one piezoelectric element for propagation of elastic acoustic longitudinal waves in the medical device system for nanovibration coating of the external and internal surfaces for preventing biofilm formation and/or dispersing biofilm formations, where the piezoelectric ceramic have at least one shape of electrodes on the internal and external surfaces, according to some embodiments of the present invention;

FIG. 5 is a schematic illustration of a process for vibration transmission as an elastic acoustic longitudinal mode (nanovibration coating) on the surface of the medical device for preventing biofilm formation and/or dispersing biofilm formations, according to some embodiments of the present invention;

FIG. 6 is a schematic illustration of excluding “dead points” achieved with a longitudinal vibration type medical device with a nanovibration coating surface, according to some embodiments of the present invention;

FIG. 7 is a schematic illustration of a medical device for preventing biofilm formation and/or dispersing biofilm formations, according to some embodiments of the present invention;

FIG. 8 is a schematic illustration of the medical device of FIG. 7 inserted inside a human body for nanovibration according to some embodiments of the present invention;

FIG. 9 is a schematic illustration of a medical device for nanovibration coating preventing of biofilm formation and/or dispersing biofilm formations showing removal and drainage of fluids from the body, as in fluid exchange, according to some embodiments of the present invention;

FIG. 10 is a schematic illustration of a cylindrical configuration piezo actuator and directions of standing waves through a fluid inside of a medical device with nanovibration coating preventing process, according to some embodiments of the present invention;

FIG. 11 is a schematic illustration of a cylindrical configuration piezo actuator wherein attached and standing waves are propagated through a hollow medical device, when fluid is drawn from the body, according to some embodiments of the present invention;

FIG. 12 is a schematic illustration of a micropressure wave propagation through an internal diameter of a cylindrical piezo actuator and a hollow medical device respectively, according to some embodiments of the present invention;

FIG. 13 are schematic illustrations of the motion effects of the vibrated surface elements due to longitudinal and bending self vibration modes of the medical device, according to some embodiments of the present invention;

FIG. 14 is a schematic illustration of a medical device for nanovibration coating preventing biofilm formation using longitudinal and bending vibration modes, wherein temporarily coinciding “dead points” are avoided according to some embodiments of the present invention;

FIGS. 15A, 15B and 15C are schematic illustrations of a medical device and diagrammatic vibration modes in a nanovibration coating preventing biofilm formation using torsion vibration modes, wherein temporarily coinciding “dead points” are avoided on surfaces of the piezo element, according to some embodiments of the present invention;

FIG. 16 is a schematic illustration of the process of vibration transmission as elastic acoustic torsion vibration modes (nanovibration coating) on the surface of the medical device for preventing biofilm formation and/or dispersing biofilm formations, according to some embodiments of the present invention;

FIG. 17 is a schematic illustration of traveling waves of different characteristics, wherein temporarily coinciding “dead points” are avoided according to some embodiments of the present invention;

FIG. 18 is a schematic illustration of a cylindrical configuration piezo actuator consisting of two parts with multiple electrodes on its surface for achieving longitudinal bending, torsion vibration modes for the nanovibration coating process, according to some embodiments of the present invention;

FIG. 19 is a schematic illustration of the ability to combine two vibration modes (1^{st} longitudinal and 1^{st} bending) for creation of motion of liquids or biological materials adjacent the device surface, according to some embodiments of the present invention;

FIG. 20 is a graph of an electrical signal based on the embodiment of FIG. 19;

FIG. 21 is a graph of velocity of the front of the wave as described in the embodiment of FIG. 19;

FIG. 22 is a schematic of elastic acoustic longitudinal traveling and torsion standing waves of various modes (nanovibration coating) on the inner surface of the medical device for preventing biofilm formation and/or dispersing biofilm formations, according to some embodiments of the present invention;

FIG. 23 is a schematic similar to FIG. 22 but illustrating wave motion on the outer surface of the medical device;

FIGS. 24 and 25 are schematics of medical devices where nanovibration waves are applied both on internal and external surfaces of a medical device;

FIGS. 26A-C illustrate multi vibration devices attached on an external surface of an elongated medical device;

FIGS. 27A-C illustrate multi vibration devices attached to an internal surface of an elongated medical device;

FIGS. 28A-G illustrate a variety of differently shaped piezo actuators for use in the present invention;

FIG. 29 is a schematic illustration of an actuator attached to a pair of connectors having different elastic physical—mechanical properties;

FIG. 30 is a schematic illustration of two connectors having different elastic physical—mechanical properties, attached to an actuator;

FIG. 31 is a schematic illustration of triple connectors having different elastic physical—mechanical properties, attached to an actuator;

FIG. 32 shows an experiment demonstrating the effect of the nanovibration coating process for preventing development of biofilms in the fluid from the medical device;

FIG. 33 shows an experiment demonstrating the effect on the medical device with nanovibration coating process for preventing the development of biofilms, according to some embodiments of the present invention;

FIG. 34 shows the results of an experiment comparing the activated versus non-activated solid medical device surface with nanovibration coating process for preventing the development of biofilms, according to some embodiments of the present invention;

FIG. 35 shows an experiment demonstrating a semisolid gelatinous material for treated and non-treated medical device illustrating the nanovibration coating process for preventing the development of biofilms;

FIG. 36 shows the experiment related to nanovibration coating process for preventing the development of biofilms, inside the urinary catheter;

FIG. 37 shows the experiment demonstrating effect on the external surface of medical device with and without nanovibration coating process for preventing the development of biofilms; and

FIG. 38 shows the scanning electron micrograph (SEM) image of urinary tract of animal contacted with non-activated catheter;

FIG. 39 shows the scanning electron micrograph (SEM) image of epithelium from urinary tract of animal contacted with activated catherer;

FIG. 40 shows at least one piezo element attached to the connector or to the hub of the standard peripheral intravenous catheter system;

FIG. 41 shows one preferred way of locating the electrical signal controller and source of energy by attaching onto a hand;

FIG. 42 shows the possibility to use one or more actuators on each of the points, which can serve as entry point for microorganisms;

FIG. 43 shows catheter, in which the actuator can be placed on the connector, on the part of the catheter which is outside of the urinary tract, on the urinary bag separately or on all of them together for the purpose of biofilm and incrustation prevention; and

FIG. 44 shows use of an endothracheal ventilation tube, which is a major cause of death due to pneumonia, resulting from biofilms.

### DETAILED DESCRIPTION OF THE INVENTION

The following description is presented to enable one of ordinary skill in the art to make and use the invention as provided in the context of a particular application and its requirements. Various modifications to the described embodiments will be apparent to those with skill in the art, and the general principles defined herein may be applied to other embodiments. Therefore, the present invention is not intended to be limited to the particular embodiments shown and described, but is to be accorded the widest scope consistent with the principles and novel features herein disclosed. In other instances, well-known methods, procedures, and components have not been described in detail so as not to obscure the present invention.

The word “biofilm” as used hereinafter may encompass microbes, microorganisms, viruses, fungi, deposits, particles, pathogenic organisms, cells, and other bioactive materials. The word “pathogenic microorganisms” as used hereinafter may encompass any organisms, including bacterium or protozoan. Such organisms may be harmful, infectious, or non-harmful.

In the description herein below, the word medical device may refer to all types of medical devices that are being used in medicine and in which biofilm prevention is a problem. Such devices may or may not be connected with the human body. Devices connected with the body are inserted or partially inserted into the body and connected with other medical devices at the same time. Another group consists of devices being only attached to the body (not inserted), for example attached to the wound. A still further group of medical devices are those that have no connection with the human body, yet still suffer from the problem of biofilm formation. An example of this situation is a test-tube for human organ synthesis. Geometrical shapes for the medical devices may be rounded or perpendicular, or any combination. Along the length the medical device may be depicted as cylindrical or in strip form, or any combination.

The process of nanovibration coating hereinafter will be clarified through explanations on the processes occurring on external, internal and torsion surfaces of medical devices, or combinations of them. Such types of medical devices include: catheters, needles, peristaltic pumps, medical tampons, bandages and others. Under the name of catheters we mean: urinary, gastric, cardiovascular, lung and others.

Nanovibration coating may be generated on the surface of a medical device by means of volumetric vibration modes. Volumetric vibration modes include longitudinal, torsion, bending, thickness, flexural, and so on, vibration modes, and superposition of these, or special wave guides.

The main feature of nanovibration coating is that every point on the surface is moving in the space of nano range (from several nanometers to tenths of nanometers). More particularly, the nano range is from about 0.001 to about 100, preferably from about 0.1 to about 50, optimally from about 1 to about 10 nanometers. It is important to say that so called “dead points” are excluded, meaning that every point of the surface is moving, and nanovibration amplitude of each point is not zero (for all space coordinates).

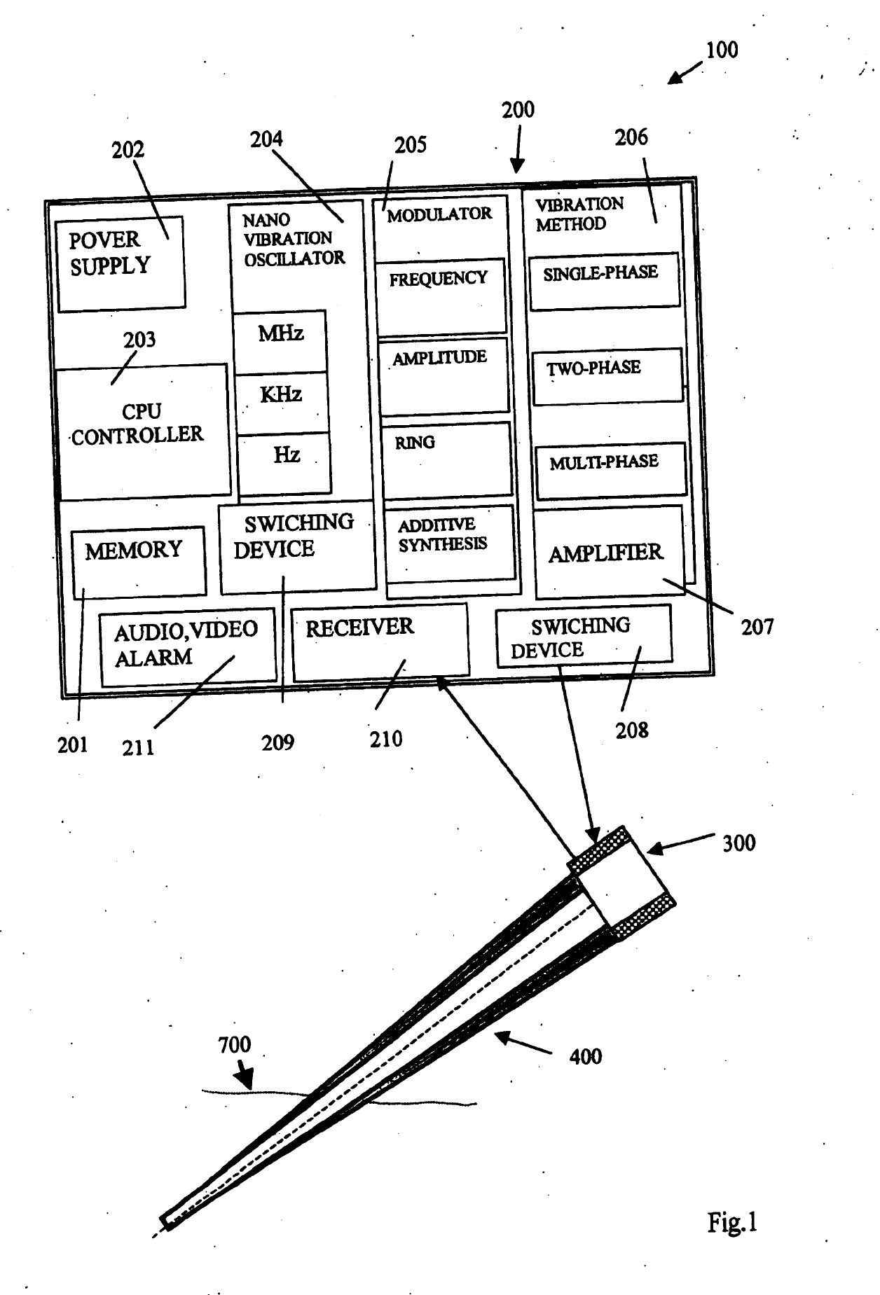

Reference is now made to FIG. 1, which is a schematic illustration of an acoustic catheter system 100, according to some embodiments of the present invention, for preventing or treating the formation of microbe colonies on a catheter, such as an IV catheter.

An acoustic medical system 100 may include a Central Processor Unit (CPU) 200 and an electromechanical energy actuator 300 directly or indirectly attachable to a medical device 400 requiring biofouling prevention. CPU 200 transmits and controls an electric signal to the electromechanical actuator. The actuator may be an electro mechanical relay and/or operate with piezomechanic or piezoelectric features. The actuator converts the electrical signal from CPU 200 to mechanical energy proportionally by range and time. As a result, actuator 300 begins to vibrate with changing energy vectors in space. To conduct volumetric mechanical vibrations in existing medical devices, one or more such actuators are attached to the medical device. The point of attachment should be chosen in such a manner that it will be possible to generate self-vibrations of the medical device 700. These self-vibrations produce the nanovibration coating over surfaces of existing (standard) medical devices.

The main medical devices have a geometric shape which may concentrate mechanical energy in a point of the body. For this reason, the electrical signal from actuator 300 is transferred to CPU 200 for control of mechanical energy pressure to biological particles. For example, diagnostic devices should not extend the range of mechanical strength to surface more than 100 mW/cm². The use of greater energy should be of shorter duration. For biofilm prevention, it is best to proceed from several hours to as long as tens of hours (long time), especially for indwelling devices, prostheses and artificial organs. More particularly, duration may range from about 0.1 to about 60 hours, preferably from about 0.5 to about 12 hours. Standard medical devices have various geometrical shapes. For example, an intravascular catheter is constructed of two geometric shapes: rounded and conical shape cylinder having an interior void of the same shape. From the point of view of mechanical energy, such shape contributes to mechanical energy concentration. As a result, mechanical energy per square unit could exceed FDA requirements. For this reason, the proposed CPU 200 should have a data input device for receiving information on the type and shape of the medical device. This information should be registered in memory block 201. CPU 200 includes a power supply 202 (battery or alternating current), memory block 201, controller 203, nanovibration oscillator 204, device for applying vibration method 205 and 206, amplifier 207, second and first switching devices 208 and 209, receiver 210 and audio-video alarm device 211. CPU 200 is connected electrically with mechanical vibration actuator 300 by forward and backward connections 301 and 302.

Generation of nanovibrations on the surface of the medical device requires exciting every point of the surface to move in the range of nanometers. The actuator 300 should receive an electrical signal from CPU 200, which should apply various (resonance and non resonance) frequencies of mechanical vibrations at the same time. It is known that materials with a piezo effect (piezomechanic or piezoelectric) can produce vibrations of different frequency resonances at the same time.

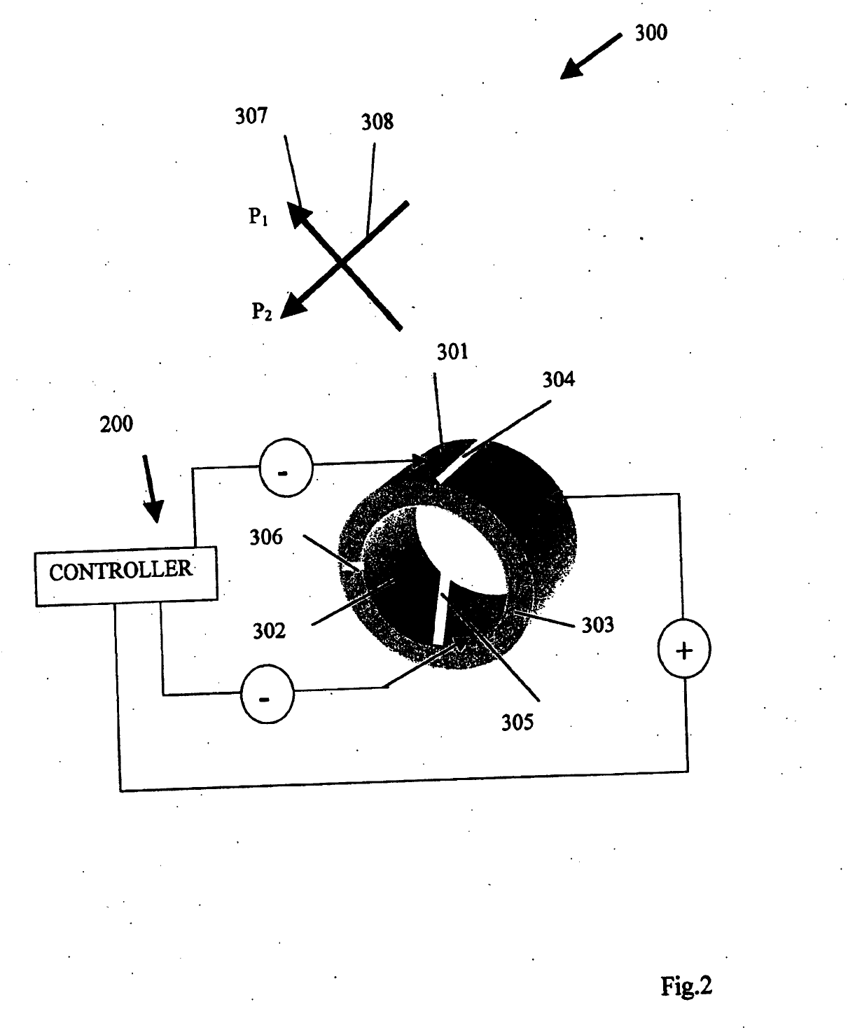

For more detailed explanations, we focus on a mechanical vibration actuator which has a piezoelement manufactured in the shape of and having more than two electrodes. FIG. 2 shows such device as a piezo cylinder formable from a single type piezo ceramics material or from combining several piezo elements. The cylinder is coated with electrodes (e.g., silver, brass, gold) and attached to electrical conductors for electrical signal transfer from CPU 200. Piezo ceramics external surfaces of connection 301, internal connection 302, and torsion 303 may be coated with electrodes. Each piezo cylinder surface electrode may have one or more isolating zones 305, 306 and these may be applied in a different manner in respect to one another. On the other hand, the piezo cylinder may have either a single or different direction of polarization (P1 or P2). FIG. 2 shows a thickness polarization 307 and length polarization 308.

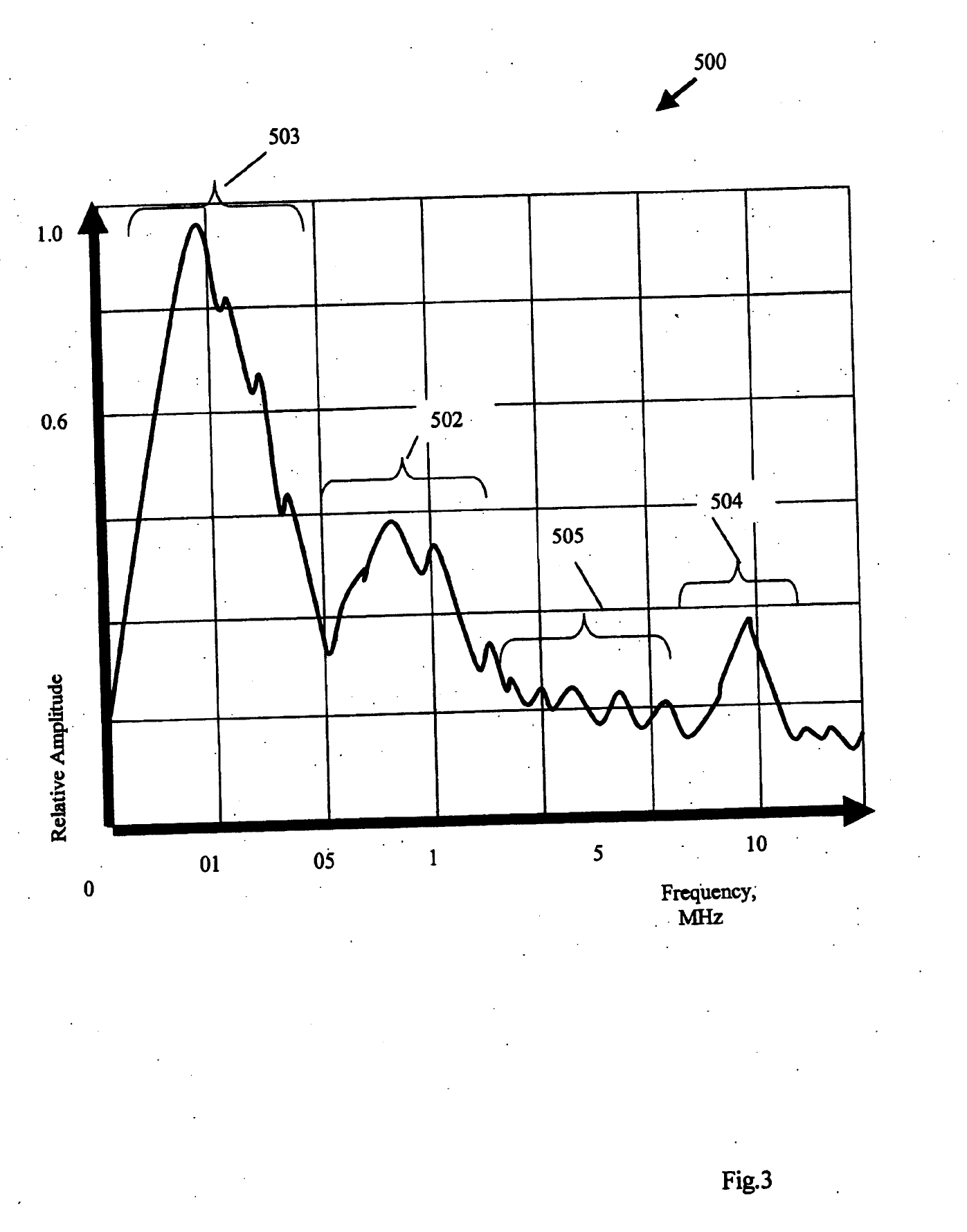

In FIG. 3, spectrum 500 is an amplitude frequency diagram for the ceramic cylinder of FIG. 2. This cylinder is made of PZT-5 material having an internal diameter of 5 mm, external diameter of 6 mm, and a length of 4 mm. Piezo ceramic elements can be manufactured so as to be capable of vibrating in a variety of modes (separately or simultaneously). For example, a piezoceramic element vibrating in a thickness mode (frequency spectrum zone 502) is capable simultaneously to vibrate in other modes (frequency spectrum zone 503). The same may occur for longitudinal, bending, torsion and other special wave modes. On the other hand, one vibration mode (for example, longitudinal) may cause other modes of vibration (thickness, bending, and so on). This feature depends on the piezo element shape, polarization direction, the lay out of electrodes on the surface of the piezo element, technical characteristics of the piezo material quality factors, and so on. The number of vibration modes depends on the shape of the electric signal applied (which may be periodical, pulse, special wave form). FIG. 3 shows zone 504 (main vibration harmonics) caused by thickness vibration mode, and the next vibration harmonics—second, third, fourth, and so on. Almost every vibration mode has not only main vibration harmonics, but also the next harmonics. The wide spectrum of frequencies acting in piezo elements simultaneously allow production of multi vibration modes in the medical devices. Under the name “system” we mean a medical device together with actuator and biomaterials in contact with all or part of the medical device. The result of this process is that each point of the surface of the medical device is moving along a three dimensional scale. This process can be called a nano vibration coating of the surface. Now we shall deal with the method to effectuate such a process.

Actuator 300 of mechanical vibrations allows production of a wide variety of mechanical vibrations ranging from several Hz to MHz, while the vibrations are excited by different phased electrical signals applied to the piezo cylinder electrodes.

FIG. 1 shows CPU 200 capable to variate the shape and time of the electrical signal. Oscillator 204 of the nanovibrations may generate separately or simultaneously electrical signals in the range of Hz, KHz, MHz. These impulses may have harmonic, impulse, or special wave forms having harmonic or non harmonic nature. Nanovibration oscillator 204 can widen the signal frequency spectrum through the first switching device 209 (which is controlled by controller 203) and modulator 205. Modulator 205 has an electronic block which separately or simultaneously can conduct modulation of amplitude (AM), modulation of frequency (FM), ring modulation (RM), additive, subtractive, gradual and wave table synthesis.

The synthesized signal from modulator 205 communicates with vibration mode device 206, which in response to a command controller 203 converts the signal to single phase, two phases, or multi phases signal. The signal through amplifier 207 and second switching device 208 is applied to different states of mechanical vibration excitement actuator 300 (for the piezo cylinder in FIG. 2 such states are generated by different electrodes).

In vitro experiments have shown the possibility to differentiate the result of the nano vibration process among different bacteria. By applying various nano vibrations to the surface of a medical device, growth of one type of bacteria may be prevented, while another type of bacteria is unaffected.

Sound or optical alarm system 211 controls and can signal when the system is operating/not operating (for example if a bad electrical contact occurs). A suitable alarm system is available under the trademark of “Uroshield” sold by NanoVibronix, Ltd. This alarm informs the user about low battery power or non contact of wires. An alarm system may also provide information on adverse non-equipment related malfunctions such as caused by the motions of the patient. These malfunctions may be excluded by an appropriate command from a sensing and adjustment element in the medical device that modulates the self vibrations of the system. This type of sensing is necessary to exclude changes perturbing parts of the medical device inside the patient's body. Internal sensing may give information on blood flow pulsation. Every mechanical vibration actuator 300 possesses a natural vibration frequency spectrum. After it has been connected to the medical device 400, we must choose the natural vibration frequency spectrum of the device. This natural vibration frequency depends upon many factors including the form of the medical device 400 and the place of attachment unto actuator 300. Therefore, feedback is important for better controlling the self vibrations in different vibration modes and their harmonics.

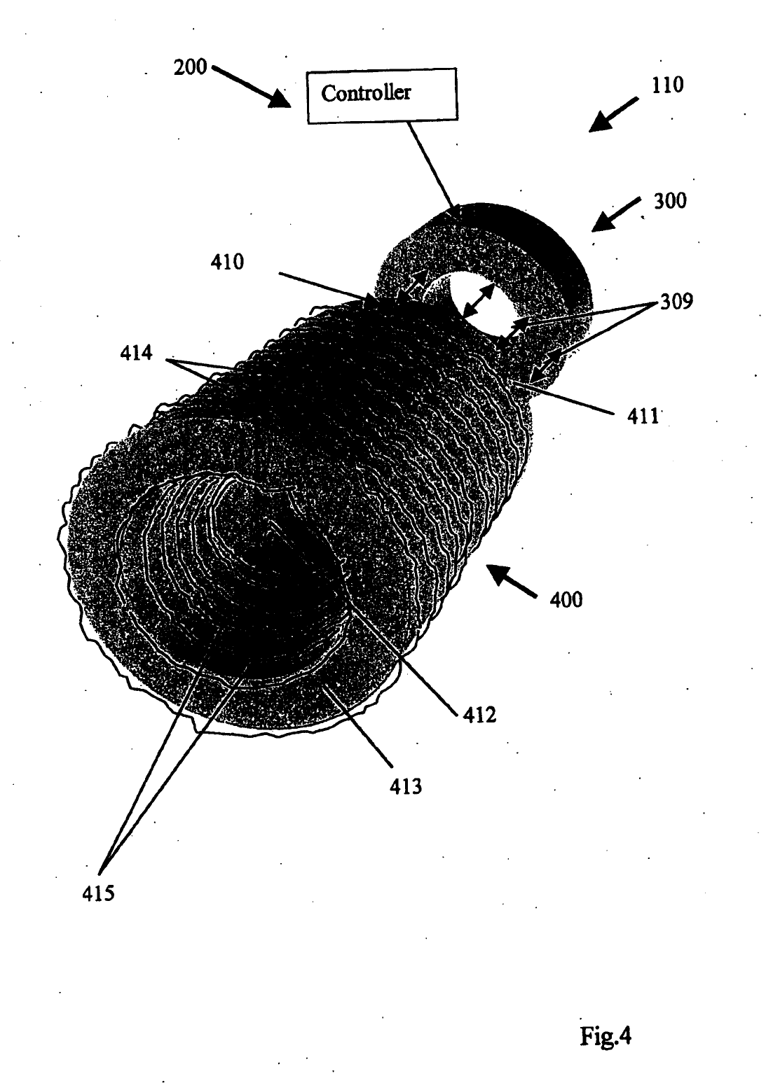

As can be seen with reference to FIG. 4, the acoustic medical device 110 includes a standard medical device part 410. Mechanical energy excitement forces 310 are shown as virtual forces excited by actuator 300 on instruction from CPU 200. Standard medical device 410 is schematically shown as a tube having external 411, internal 412 and torsion 413 surfaces. Mechanical vibration actuator 300 excites part 410 to vibrate self spatial multi vibrations in x, y, z coordinates. As a result, elastic waves of mechanical vibrations (in the range of nano scale) are excited on internal 412 and external 411 surfaces. Due to the CPU 200 and mechanical vibration excitement actuator 300, it is possible to simultaneously create elastic vibrations on every surface point in the x, y, z coordinates (the scale of time can change from micro seconds to parts of seconds). These elastic mechanical nano scale vibrations that include simultaneously external and internal 415 elastic vibrations can propagate as chaotic running surface elastic waves in different x, y, z coordinates. The vibrations of such type simultaneously with external and internal surface vibrations can also be excited on the torsion surface (not shown in FIG. 4). The vibration of such a type, excited on the surfaces of existing (standard) medical devices, is the so-called nanovibration coating. It is possible to create nanovibration coating processes separately on external or on internal surfaces, or alternately, by choosing or changing the point of attachment of the mechanical vibration actuator 300, direct the range and character of vibrations. More specifically, the different surfaces may be coated by vibrations separately, simultaneously or alternately. Control of the range and character of the nano vibration coating process depends on mechanical—physical characteristics of the medical device. On the other hand, the process depends on acoustical impedance of the system, consisting of the standard medical device and mechanical energy actuator. Acoustic impedance is the reflection and transmission of mechanical energy at a boundary between two media. Nanovibration coating on the separate medical device surfaces can be controlled by selection of different acoustic impedances at the contact zone between mechanical energy actuator 300 and standard medical device 400 by means of matching materials with different acoustic characteristics.

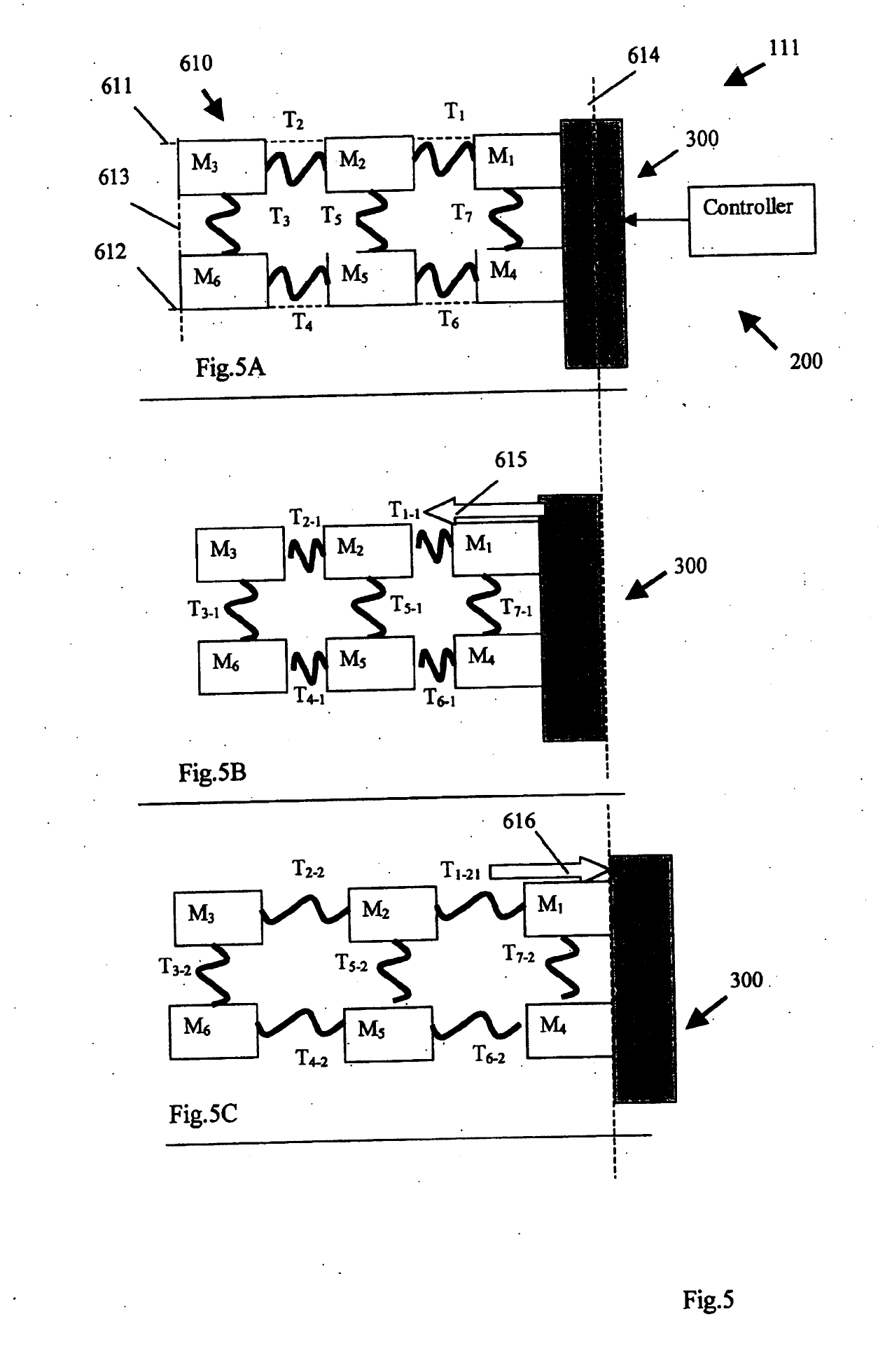

FIG. 5 is a schematic view of the nanovibration coating process on a standard medical device surface, when the volume of the medical device is excited to longitudinally vibrate. The figure illustrates a standard medical device 610, which is conditionally divided between external 611, internal 612 and torsion 613 surfaces. These surfaces are conditionally made of small masses M₁,M₂,M₃,M₄,M₅, and M₆. Conditional damper-spring systems T₁,T₂,T₃,T₄,T₅,T₆, and T₇ exist between these masses. Masses M₁ and M₄, which have a damper-spring system T₇, are tightly attached to mechanical vibration energy actuator 300 that converts electrical signals coming from CPU 200 into vibrations. Conditional coordinate for mechanical vibration energy is 614. External surface 611 of standard medical device 610 may conditionally consist of masses M₁, M₂ and M₃, and have corresponding damper-spring systems T₁ and T₂. Internal surface 612 of this device consists conditionally of masses M₄, M₅ and M₆, having corresponding damper-spring systems T₄ and T₅. Torsion surface 613 of this device consists conditionally of masses M₃ and M₄, having corresponding damper-spring system T₃. While applying an electrical signal from CPU 200 to mechanical vibration source actuator 300, the mechanical movement is excited in a perpendicular direction 615 respective to coordinate 614 (FIG. 5b). At the same time, the device is excited to vibrate in a longitudinal mode. When mass M₁ is moving in direction 615, there is a change in the dynamical characteristics of the damper-spring system. As a result, mass M₂ moves differently in respect to mass M₁. The dynamical characteristics of the damper-spring system is illustrated as T₁₋₁, T₁₋₂ . . . T₁₋₇, when piezo actuator is moving in direction 615, and as T₁₋₂, T₂₋₂, T₃₋₂ . . . , T₇₋₂, when the direction of piezo actuator is 616. The mass M₂ transmits the movement to mass M₃ due to spring damping system T₂₋₂, and the mass M₃ is moving differently in respect to the mass M₂. Masses M₁, M₂, M₃ illustrate conditions of the external surface. These masses are bound through spring-damping systems T₇₋₁, T₅₋₁, T₃₋₁ with external surface masses M₄, M₅, M₆. Under ideal conditions masses M₁ and M₄ are moving equally, and the torsion surface masses M₃ and M₆ are moving in the same manner. Under actual conditions these movements are different. When an electrical signal applied from CPU 200 has an opposite polarity (as shown in FIG. 5c), the device vibration direction is 616. That means external masses M₁, M₂, M₃ and internal masses M₄, M₅, M₆ are moving in direction 615, and afterwards in direction 616. In such a manner, the elastic waves are moving on the surface. The character of these waves depends on the range of longitudinal mode harmonics, medical device elastic properties, form, material density, and mechanical energy oscillations frequency. As a result, longitudinal type mechanical vibration energy excites the vibration of micro masses on the external and internal surfaces. The nanovibration coating process on external and internal surfaces will be stable under conditions where the excited longitudinal vibrations (for example, excited by piezo element thickness vibration mode) match to the natural system vibration, and device 400 is vibrating at the resonance.

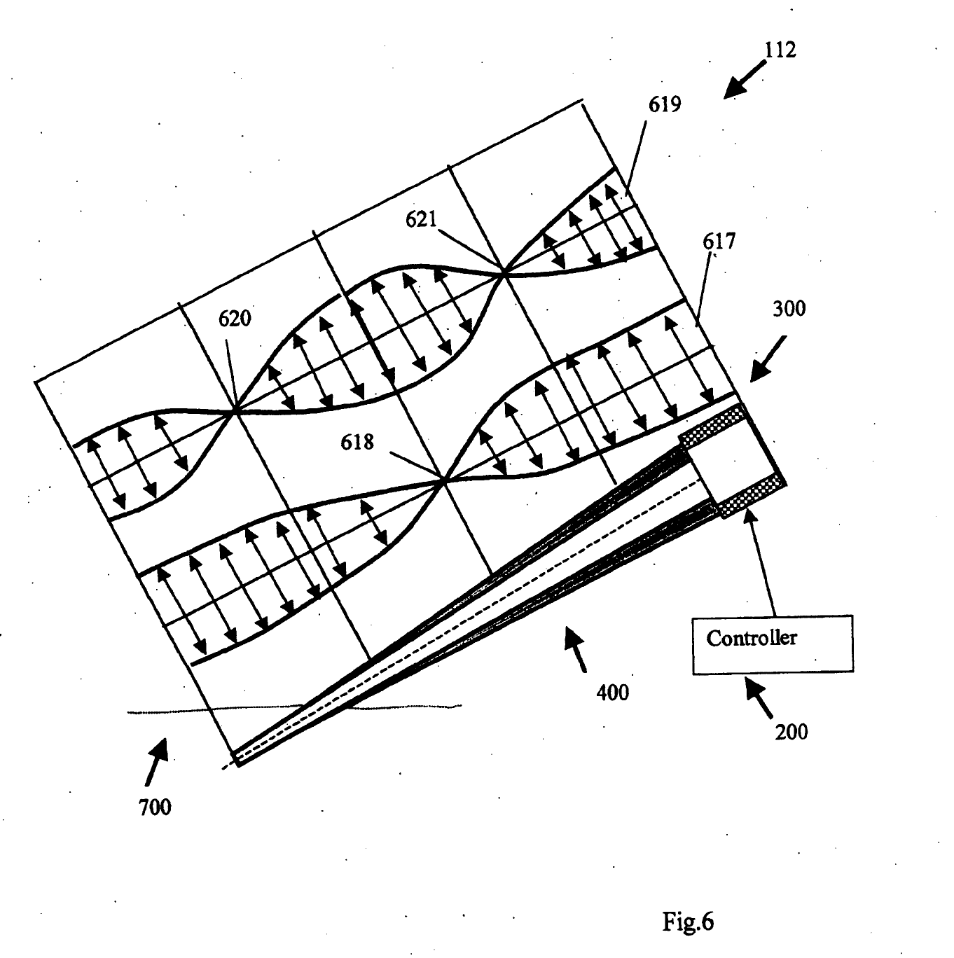

FIG. 6 illustrates a standard medical device with a biofilm treatment system, according to some embodiments of the present invention, wherein longitudinal vibration energy is transmitted through external and internal surfaces of the device. Applying two or more mechanical vibration modes 617 and 619 simultaneously, it becomes possible to achieve a situation where every material point on the external and internal surface of the device is moving. In-vitro and in-vivo experiments conducted in our laboratories, show that the nanovibration coating process prevents biofilm formation on the surfaces of medical devices, or at least considerably decreases biofilm. The internal surfaces of medical devices can be used for two purposes. One purpose is to eliminate liquids from biomaterial or from the human body, the other is to introduce liquids and semi liquids into the body.

FIG. 6 illustrates a biofilm treatment system according to some embodiments of the present invention having the simplest longitudinal vibration character. The device 112 is in tube form, open on both ends and vibrates in longitudinal half resonance. The device is manufactured of solid material (metal, plastic, silicone, rubber and so on) which allows creation of transverse mechanical vibration energy. The direction of this energy is perpendicular to the direction of longitudinal mechanical vibrations. While nanovibration coating process is achieved by longitudinal vibration type, the combination of more than one harmonic mode 617, 619, enabling avoidance of “dead points” 618, 620, 621 (which are inevitable while using one vibration mode), and at no time will the vibrations be zero (by amplitude, frequency, plane). In order to avoid “dead point,” two different longitudinal vibration modes are made to not coincide, as it is shown in FIG. 6.

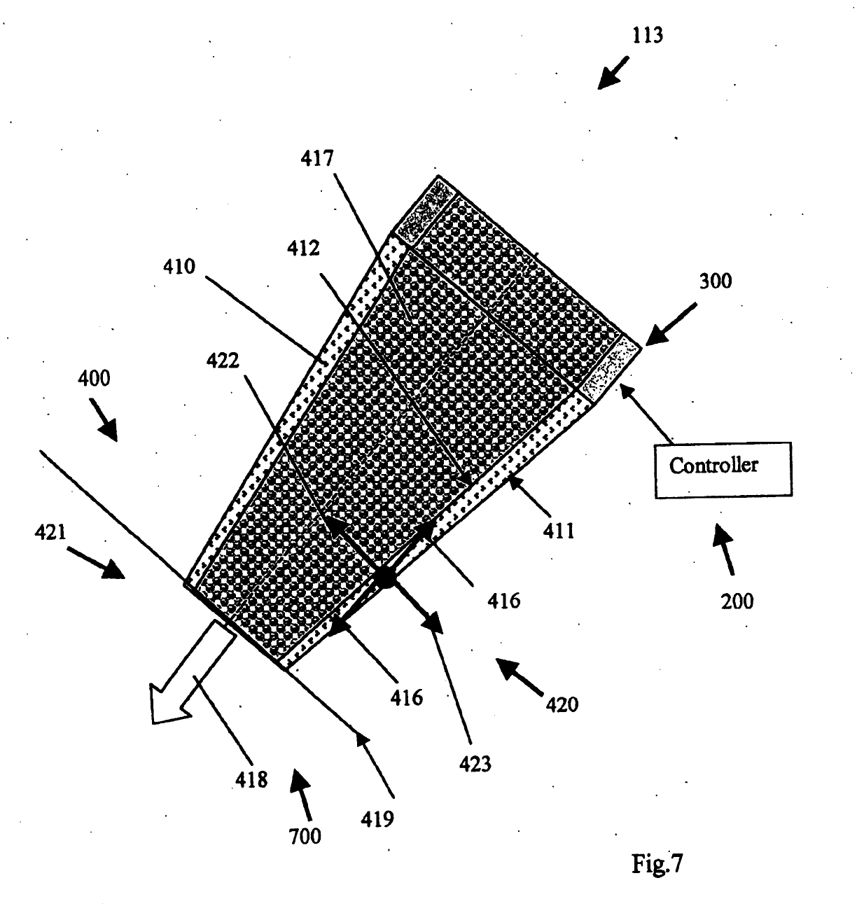

FIG. 7 illustrates part 113 of the acoustic medical device wherein on an internal surface of the part 113 is generated the nanovibration coating process. This process is created by means of electro mechanical actuator 300 (for example, by means of piezo cylinder). Actuator 300 is tightly attached to standard medical device 400 illustrated by FIG. 6. Resonance longitudinal mechanical energy is created by this system. Energy in the form of waves is transferred in the direction 416. Liquid 417 is pumped in direction 418 through internal surface 412 of the medical device. External surface of medical device may be conditionally divided by line 419 into two parts. Right part 420 on an external surface 411 is intended to remain outside the human body. Left part 421 on the external surface 411 in intended for insertion into the human body (or biological media), which allows transmission of elastic mechanical vibrations. Internal surface 412 remains in contact with the flow of liquid 417. As a result, the created nanovibration surface coating on the internal and external surfaces of standard medical device 400 has different mechanical energy propagation character. Energy propagation will depend on elastic-mechanical properties of the material which is in contact with the nanovibration coating process.

The coating process achieved (due to longitudinal vibration energy) on internal surface 412 is transferred through the material of the device in the direction 416 of these vibrations. The internal surface 412 is in constant contact with the liquid flowing in the direction 418. This factor excites the nanovibration coating process by transferring mechanical vibration energy perpendicular (transverse) to direction 416. Ordinarily, the transverse mechanical energy in the direction 422 has a detrimental effect on biofilm formation. Therefore, it must be controlled and not extend beyond 100 mW/cm2. On the other hand, such transverse energy phenomenon may sometimes be of value. Homogenization may be one benefit. It must be said that the additional (transverse) energy is much smaller in comparison with coating process energy.

FIG. 7 illustrates the transverse mechanical energy, in the form of elastic waves, being transmitted from external surface 411 of the device 400 in a direction 423. From external medical device surface portion 420, the transverse energy is transmitted in the direction 423. Transverse energy has no effect on the portion of the medical device which is held outside the body because the air is a poor transmitter of acoustical energy. Consequently, the mechanical vibration source can be attached even from a distance, and provided without considerable loss of energy through a length of the device.

At the portion of the device which is inserted into the body, the transverse energy effects nearby tissues along direction 423, and in such a way the nanovibration coating process reduces biofilm formation. More precisely, the biological mechanism could be described as follows. When a foreign medical device is inserted into the body, in the nearby surfaces of transitional epithelium, some biological processes occur including encrustation, increase in pH, stagnation and epithelial shedding. These phenomenon lead to a new physical barrier formed from encrustation of dead cells, minerals and exudates, in which bacteria multiply and lead to infection. The known technologies, for example, those using coating by disinfectant, antibiotics and silver ions, do not overcome this problem. The active ions and molecules of the coating cannot penetrate or cross the barrier behind which the bacteria are sheltered, build the biofilms and penetrate the mucous membrane itself, leading to the known complications. Nanovibration coating technology, on the other hand, overcomes the described obstacles as demonstrated by experiments conducted in our laboratory. The transverse energy nanovibrations are being transmitted in the direction 423 and are preventing negative biological processes.

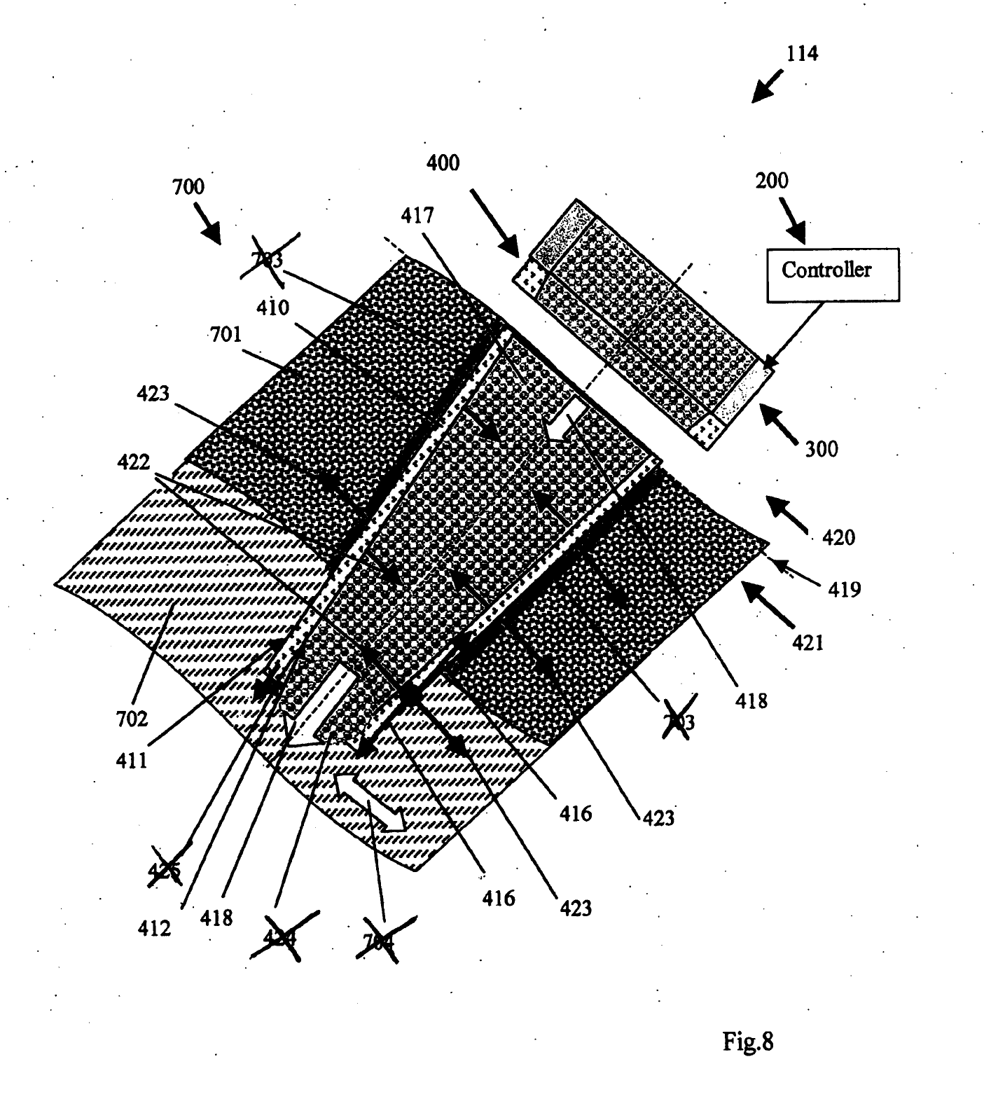

FIG. 8 illustrates the portion 410 of acoustical medical system 114 which is inserted into the body 700. Portion 410 of medical device 400 is inserted into the body (for example, blood artery 702) through biological tissue (skin) 701. A medicine or biological liquid 417 is conveyed through internal surface 412 in the direction 418 to blood artery 702. To create nanovibration coating process on the internal 412 and external 411 surfaces of the standard medical device 400, the source of mechanical vibrations actuator 300 should be attached to this device. The mechanical vibrations source actuator 300 is controlled by electrical signals applied from CPU 200. Line 419 conditionally divides medical device 400 into two parts 420 and 421. The effects of the nanovibration coating processes acting on part 420 have already been discussed above with respect to FIG. 7. FIG. 8 further illustrates part 421 inserted into the human body. The nanovibration coating process terminates at wall 410 of the device. The frequency of this process is in the range KHz to MHz, generally from about 10 KHz to about 100 MHz, and optimally from about 4 MHz to about 80 MHz. More particularly, where one vibration mode is applied separately, the preferred frequency ranges from about 0.01 to about 0.5 MHz. A simultaneous combination of two vibration mode has frequencies preferably ranging from 0.1 to about 10 MHz, with a variety of amplitudes. The acoustic energy of mechanical vibrations is less than 10 mW/cm2, preferably less than 1 mW/cm2 and may range from 0.001 to less than 10 mW/cm2. As explained earlier, every point along the surface (external, internal, torsion) is excited to vibrate in nano scale. The frequency of these vibrations is variable in time and dependent on the type of mechanical vibration excitement actuator and inherent natural vibration of the medical device system.

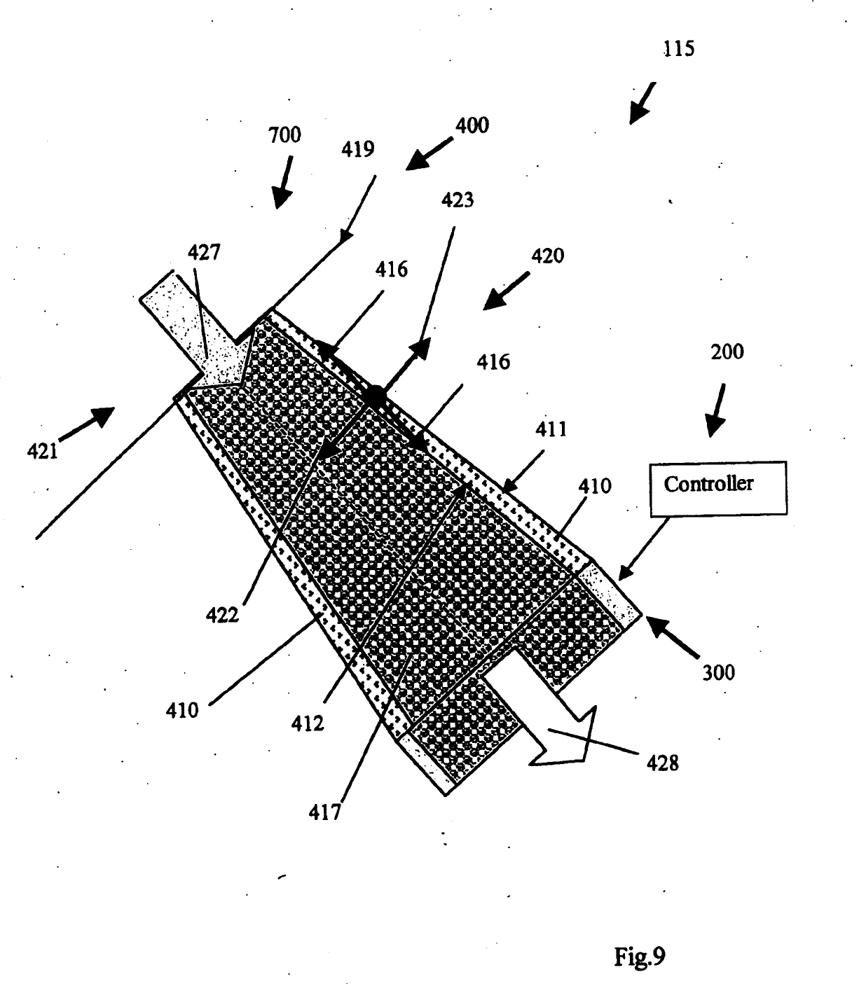

FIG. 9 illustrates the part 410 of acoustical medical system 115 which exits from the body 700. The nanovibration coating process is created on the part of medical device 420, which in this case is the part through which liquids are thrown out of the body (for example, catheter tube). The liquid 427 enters part 410 of device 400. The liquid exits the medical device via an opening in direction 428. Nanovibration coating process applied in direction 428 has several advantages: this process reduces dynamic friction between flowing liquid and the medical device surface, the problem of blocking is solved and the drawing of body fluids is speeded.

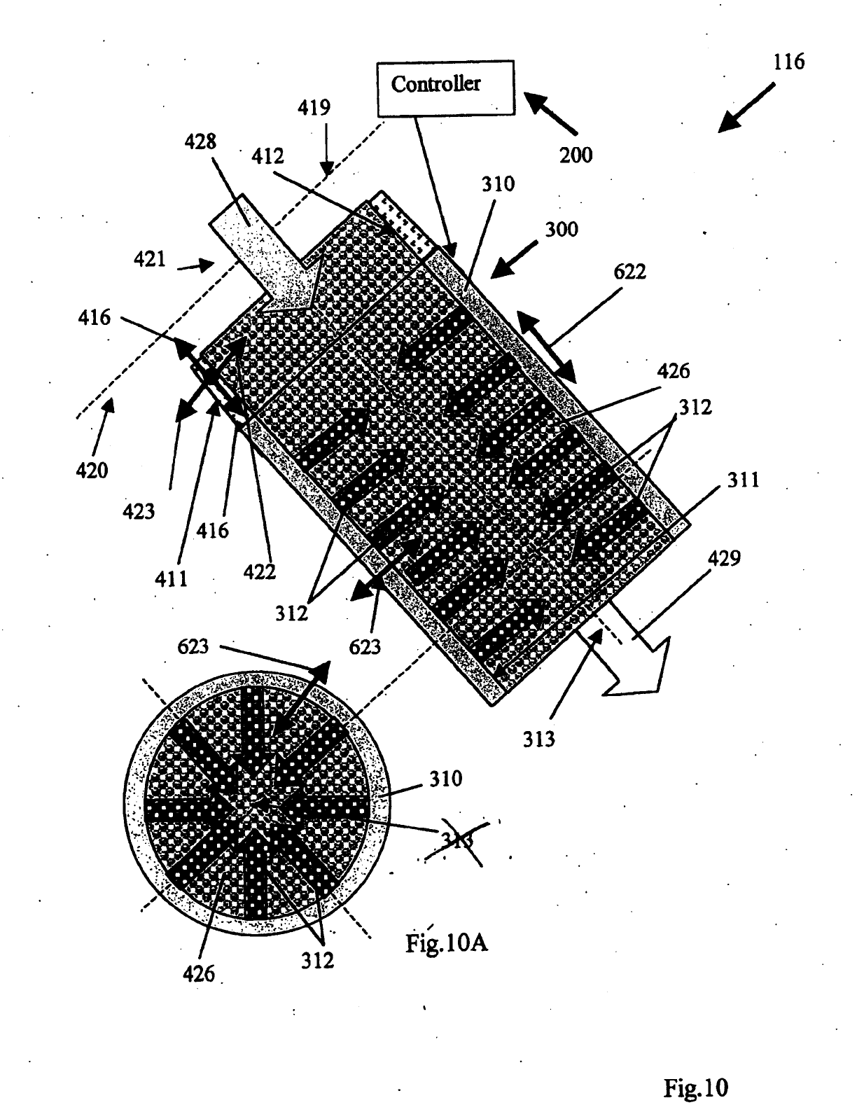

FIG. 10 illustrates the view 116 of a cylindrical piezo ceramic configuration 300 attached to medical device 400. Through the inner channel of medical device the liquid 426 enters and follows to the exit through the inner channel of piezo ceramic cylinder 310. Piezo cylinder 310 generates vibrations adjusted by controller 200, and creates standing waves in the liquid 427 (FIG. 10A). This may be achieved as a result of vibration of the walls of piezo cylinder 310 in thickness mode 623. The variety of micro pressures in direction 312 are created, and focused the center of the cylinder. These pressures may change the biological matter within and quality of the exiting liquid 429. This effect is achieved simultaneously combining thickness, bending and torsion vibration modes of the cylindrical piezo ceramic element. All possible shapes of piezo elements can be used. Non-limiting examples include tube and cone shapes.

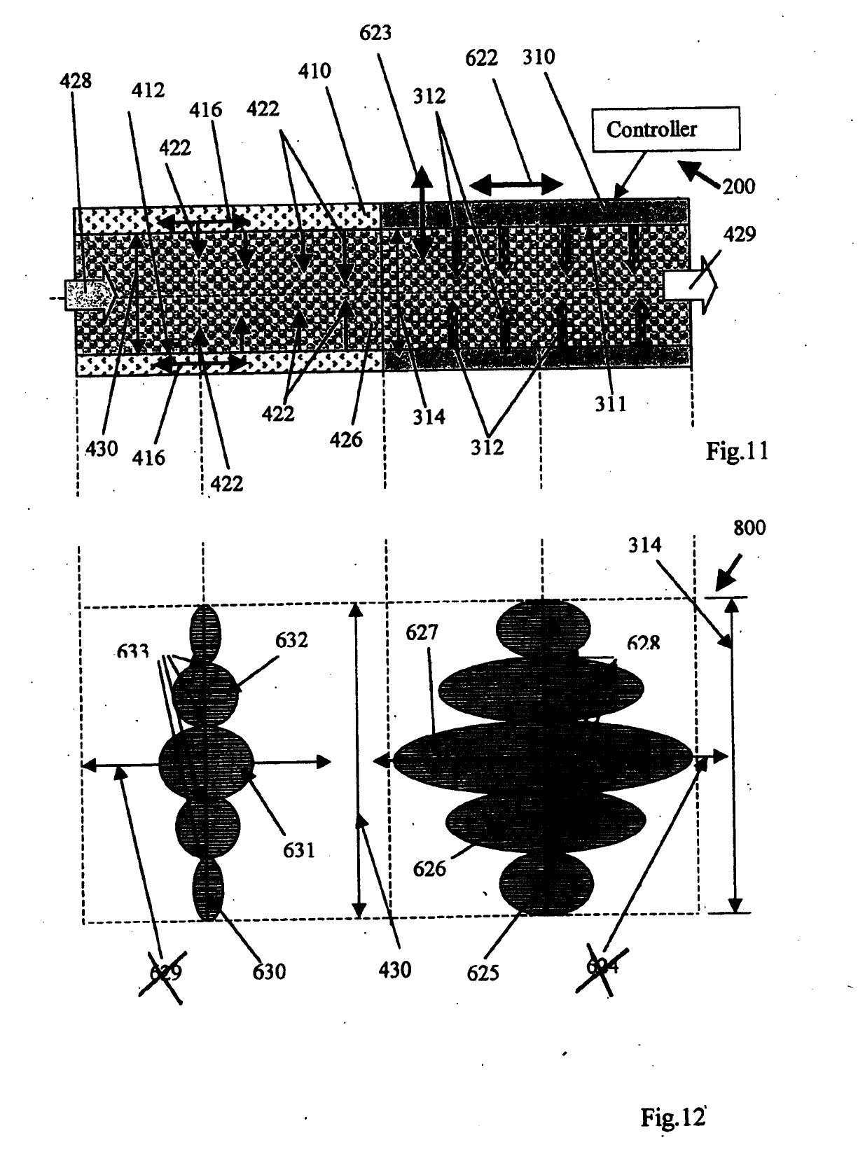

FIGS. 11 and 12 illustrate the view 800 of the effect of standing waves in the channel of a medical device attached to a cylindrical piezo element. Longitudinal 622 and thickness 623 vibration modes are illustrated therein. FIG. 11 shows that the effect of killing and preventing bacteria in the liquid extends outward a certain distance from the cylindrical piezo element in both directions of the longitudinal axis. FIG. 12 illustrates a diagram of pressure forces on the liquid. Herein is shown a piezo element 300 of cylindrical shape with inner diameter 314 and medical device 400 of inner diameter 430. The character and quality of standing waves that are produced by the cylindrical piezo element depends on piezo element oscillations frequency as well as on mechanical and acoustic parameters of the system consisting of piezo element and liquid. The distribution of micro pressures of standing waves in the inner channel is shown in FIG. 12. These waves include maximum amplitudes 625, 626 and 627 and minimum amplitude of zero at the point 628 (the point where pressure does not exist). Additional piezo element vibration modes are applied (e.g., bending mode) to avoid “dead points,” where pressure is zero. Dead points 628 are constantly changing their location. By this mechanism, we achieve the inventive concept of the coating process, i.e., having no “dead points” of vibration amplitudes. The same effect is achieved in the inner channel of the medical device, while applying bending, torsion and longitudinal elastic waves to inner surface 410. These vibrations excite standing waves 630, 631 and 632 in the liquid. By combining elastic waves propagation character, we can avoid “dead points” 633, by changing their place and time. The experimental data (NanoVibronix, Inc.) shows that micro pressures in the liquid in contact with medical device may be in the range of tenth atmospheres, and in the liquid in contact with piezo element—even hundreds of atmospheres (at very short time moments). As the direction of these micro pressures is changing 0.1-1 million times/s, an effective bacteria killing process is achieved, avoiding “dead points.”

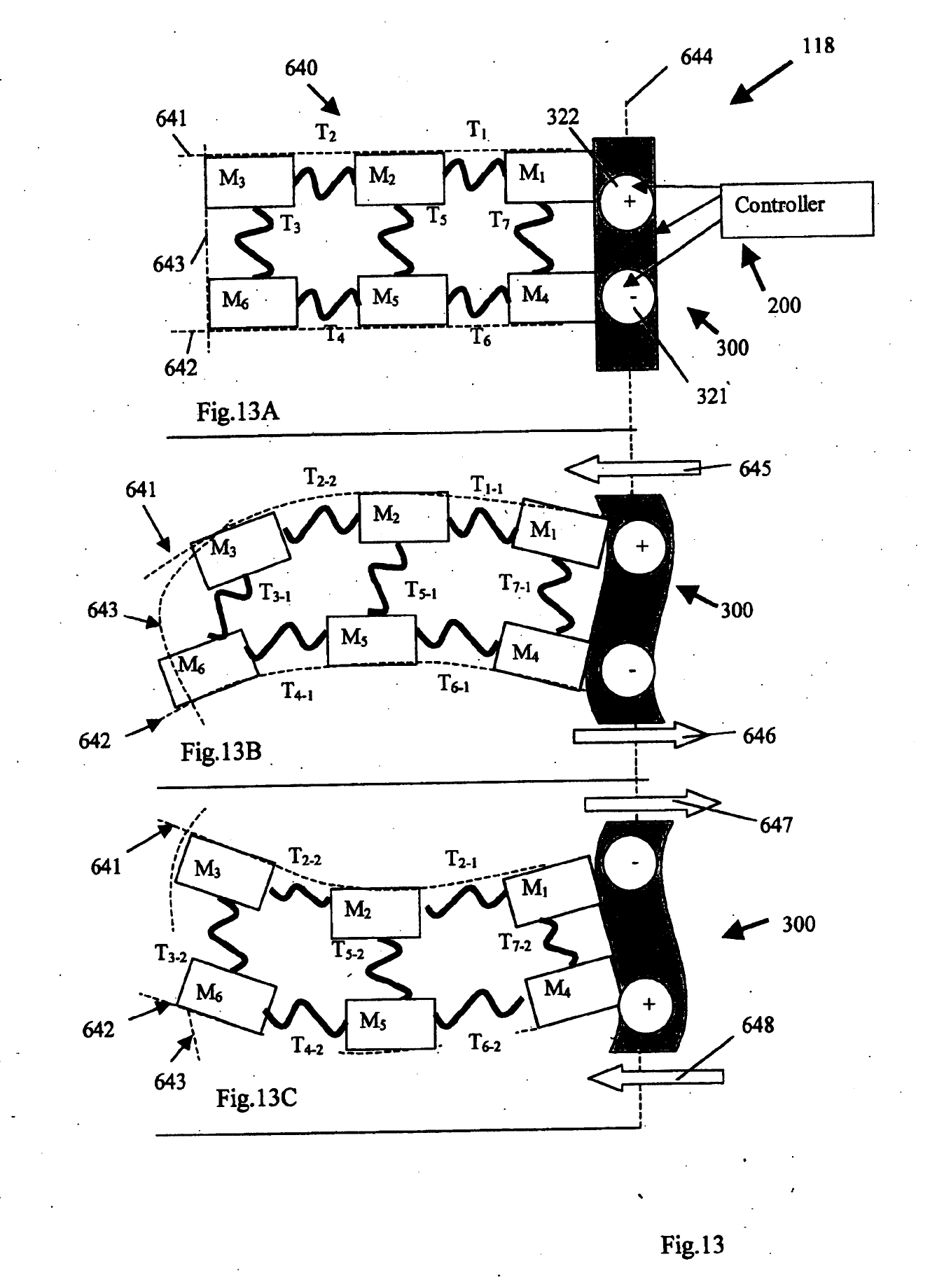

FIGS. 13(A, B and C) illustrates the view 118 of schematic illustrations of nanovibration coating process on standard/or special medical device surface, while the complete volume of the medical device is excited to vibrate simultaneously in longitudinal and bending modes. FIGS. 13(A, B and C) conditionally illustrates medical device 640, which has external 641, internal 642 and face 643 surfaces. These surfaces conditionally consist of small masses M₁, M₂, M₃, M₄, M₅, and M₆. Conditional damper-spring systems T₁, T₂, T₃, T₄, T₅, T₆ and T₇ exist between these masses. The masses M₁ and M₂ on the surface of the medical device are tightly attached to mechanical vibration energy actuator 300, which converts electrical signals coming from CPU 200 into vibrations 614, the latter being a conditional coordinate of medical vibrations.

An external surface 641 of medical device 640 may conditionally consist of masses M₁, M₂, and M₃ and have corresponding damper-spring systems T₁ and T₂. An internal surface 642 of this device conditionally consists of masses M₄, M₅ and M₆, having corresponding damper-spring system T₄ and T₅.

The torsion (face) surface 643 of this device conditionally consists of masses M₃ and M₆ having corresponding damper-spring system T₃. Mechanical vibration energy source actuator 300 (e.g., piezo element) has electrodes 321 and 322, each with different direction of polarization of piezo material. While applying an electrical signal from CPU 200 to the mechanical vibration energy source (piezo element's 300 electrodes 321 and 322), mechanical movement is exited in perpendicular directions 645 and 646 respective to coordinate direction 644 (FIG. 13B). The mechanical device is therefore excited to vibrate in longitudinal and bending vibration modes simultaneously.

In such a manner, mass Ml moves in a curvilinear direction between coordinate directions 644 and 645, and results in changes of dynamical characteristics of the damping-spring system. External surface 641, internal surface 642 and face surface 643 move in complicated trajectories between directions 644 and 645. The generated nanovibration surface coating as a result of damping-spring dynamical characteristics will cause all surface points to move simultaneously with different amplitudes in two coordinate directions 644 and 645. It may be concluded that all the points of the surface simultaneously are moving accordingly to three coordinate directions 644, 645 and 646 (complicated curve) with different energies, because of damping-spring effect.

FIG. 13C shows, that after half a period of vibrations, the movement vector changes by 180°, the direction 645 is changed to direction 647, and direction 646 is changed to 648. The changes in vibration direction are directly proportional to the frequency of longitudinal and bending vibration modes.

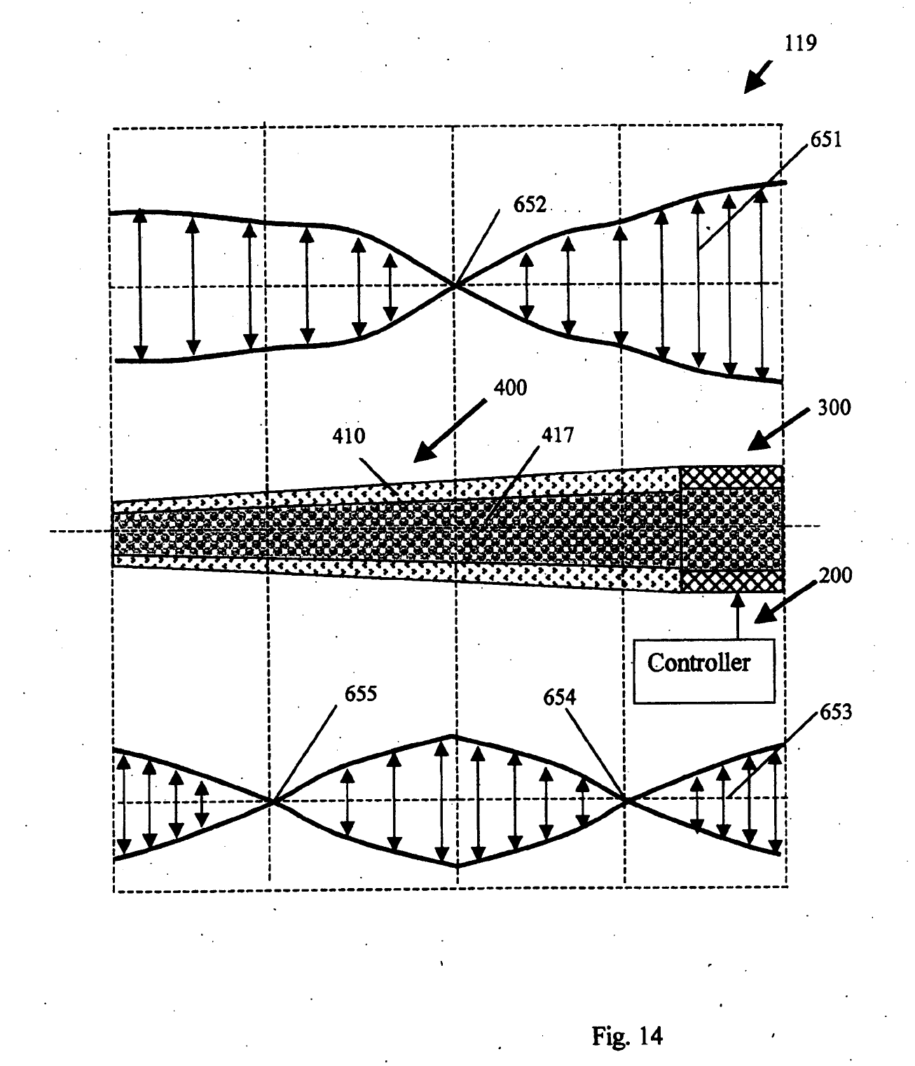

FIG. 14 illustrates nanovibration coating process 119, which may be created by combining the basic vibration modes (longitudinal and bending) together with natural vibration modes of different harmonics (1,2,3,4 modes). This may be achieved by manipulating with piezo element electrode configurations and by changing the phase of electrical signal.

FIG. 14 illustrates in diagrammatic form a vibration process 119. Here, the surface of the medical device 400 is vibrating in basic bending mode 651 and in the third vibration harmonic 653 simultaneously. Vibrations 651 and 653 display “dead points” 652, 655, 654 which do not coincide (i.e., the points where amplitude is 0). By eliminating coincidence of dead point areas, the problem of biofilm pockets is overcome. The same objective may be achieved, while applying as the basic vibration mode a longitudinal or torsion type, a combining of these vibration modes with different harmonics. The basic vibration mode type is considered in relation to medical device geometrical shape and acoustic parameters, the mobility required and the size of the electrical source.

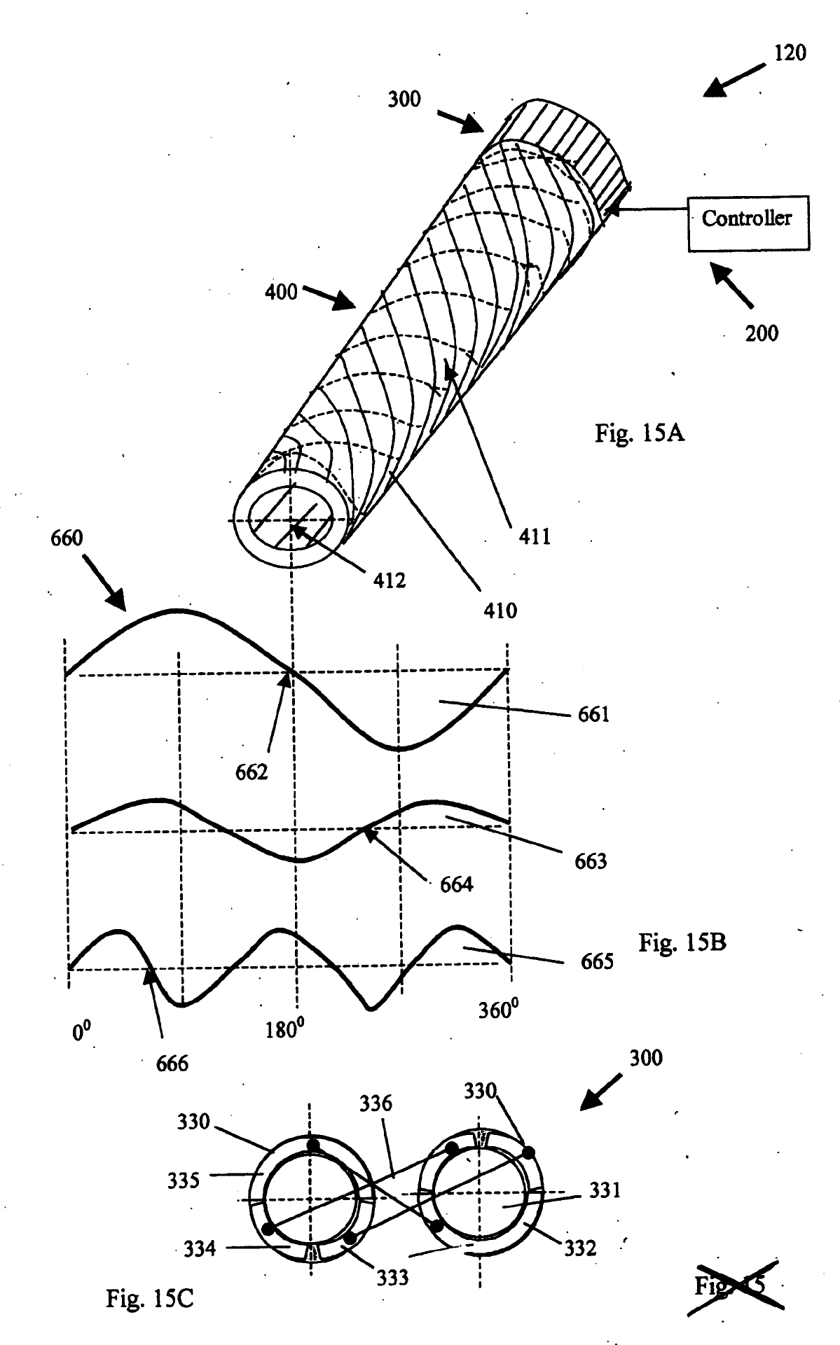

FIGS. 15(A, B, C) illustrates the nanovibration coating process 120, which is achieved by means of torsion vibration mode in the medical device 400 in combination with different torsion vibration harmonics 661, 663 and 665. The problem of “dead points” 662, 664 and 666 is solved by combination of more than one harmonic mode. At no time will vibrations of external 411 and internal 412 surfaces be of zero amplitude. Piezo element configuration for torsion vibration mode oscillations is shown in FIG. 15C. The piezo element 330 has a ring configuration and an inner cavity 331 may be used for liquids to exit or to enter. The piezo element 330 has electrodes 333 on inner and outer facial surfaces. These electrodes are divided into different groups 332, 334 and 335, which are electrically connected by wires 336. One phase electrical signal is applied from CPU 200 to the wires 336 of the electrodes. This signal excites torsion vibration in piezo ring 330, and the vibrations are transferred to the mechanical device.

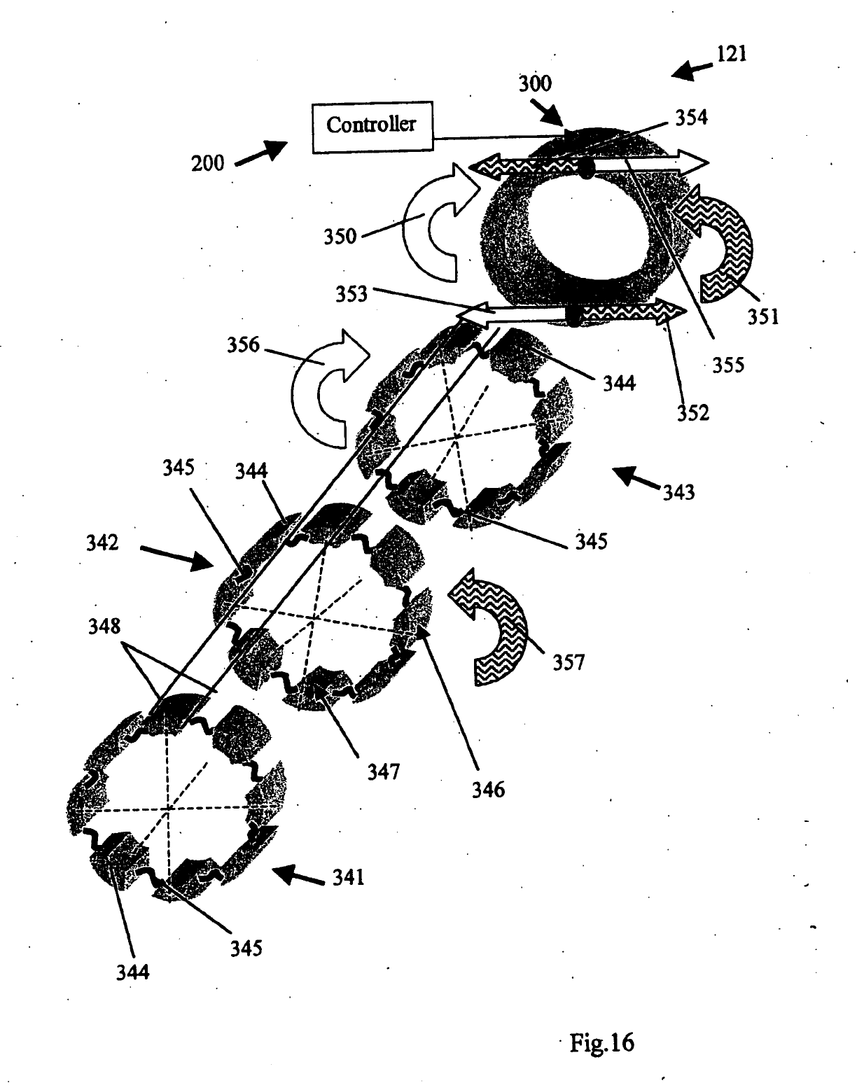

FIG. 16 illustrates view 121, which is a schematic explanation of the nanovibration coating process on a medical device surface, while excitement is via torsion mode. The piezo element 300 has a ring configuration, similar to those shown in FIG. 15C, and excites a torsion movement 350 forward and 351 backward. These movements are illustrated as directional arrows 352, 354 and 353, 355 for one conditional mass 344. While the piezo element is vibrating in torsion directions 350 and 351, the energy of these vibrations is transferred to the medical device. The inner and outer surfaces of the device are excited to vibrate in directions 356 and 357, and, as a result, nano amplitude vibrations are realized.

Under the assumption that the medical device has an appropriate size, it may be considered that the medical device consists of several conditional rings 341, 342 and 343. These are bound together with a damping-spring system (which is not shown). Each conditional ring consists of conditional masses 344, which have conditional damping-spring systems 345 between them. When the medical device is excited to vibrate in torsional mode, the masses 344 in the conditional rings are vibrating with different phases, because of different damping-spring system characteristics. The lines 348 show repartition limits of conditional masses. Our experiments have proven that torsional vibrations provide good results against heavy biofilms and bacterial encrustations, by means of the presently disclosed nanovibration coating process. Nonetheless, such process is difficult to apply because of high energy consumption.

FIG. 17 illustrates the medical device 122. Depicted on internal 412 and external 411 surfaces thereof is the short-term nanovibration coating process of this invention. This process has a direction vector and it is achieved by applying short-term electrical impulses to piezo elements. The piezo element 300 with hollow cavity is vibrating in the direction 360. The magnitude of these vibrations is controlled through CPU 200, which applies short term electrical impulses, and they excite stroke mechanical vibrations in the piezo element, which are transferred to the medical device. The impulse signals are matched to have the third harmonic shape, this impacts to simultaneously obtain a basic vibration mode and a strong third harmonic vibration mode. The shape of the vibrations that pass to the medical device are depicted as mode 661 and third harmonic mode 662. Vibration 663 is a summary distribution of amplitudes of 661 and 662 and graphically illustrates the nanovibration coating process in medical device. By actuating different vibrations, combinations of vibrations modes can be created simultaneously and changed periodically. All of these vibration modes may be achieved through a single piezo element. Nanovibration coating processes may be activated by pulsed vibration type (intermittent) according to desired applications for energy optimization (FIG. 17).

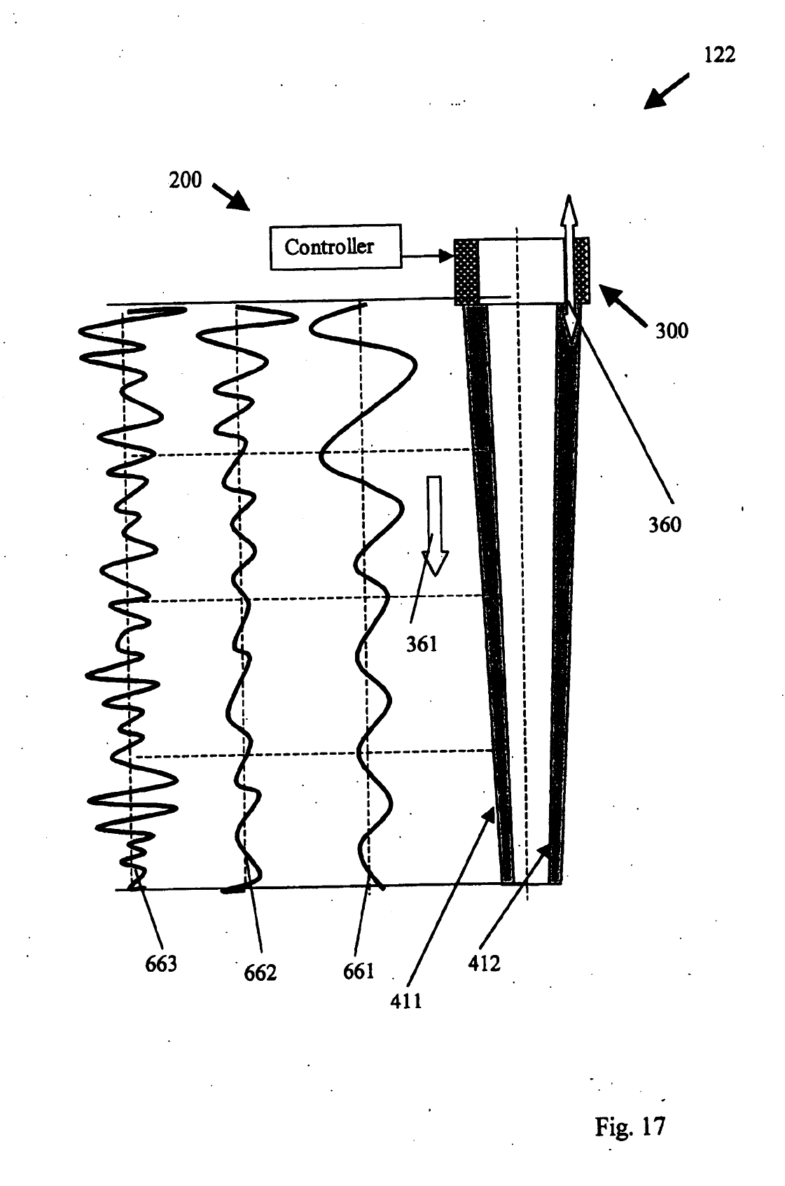

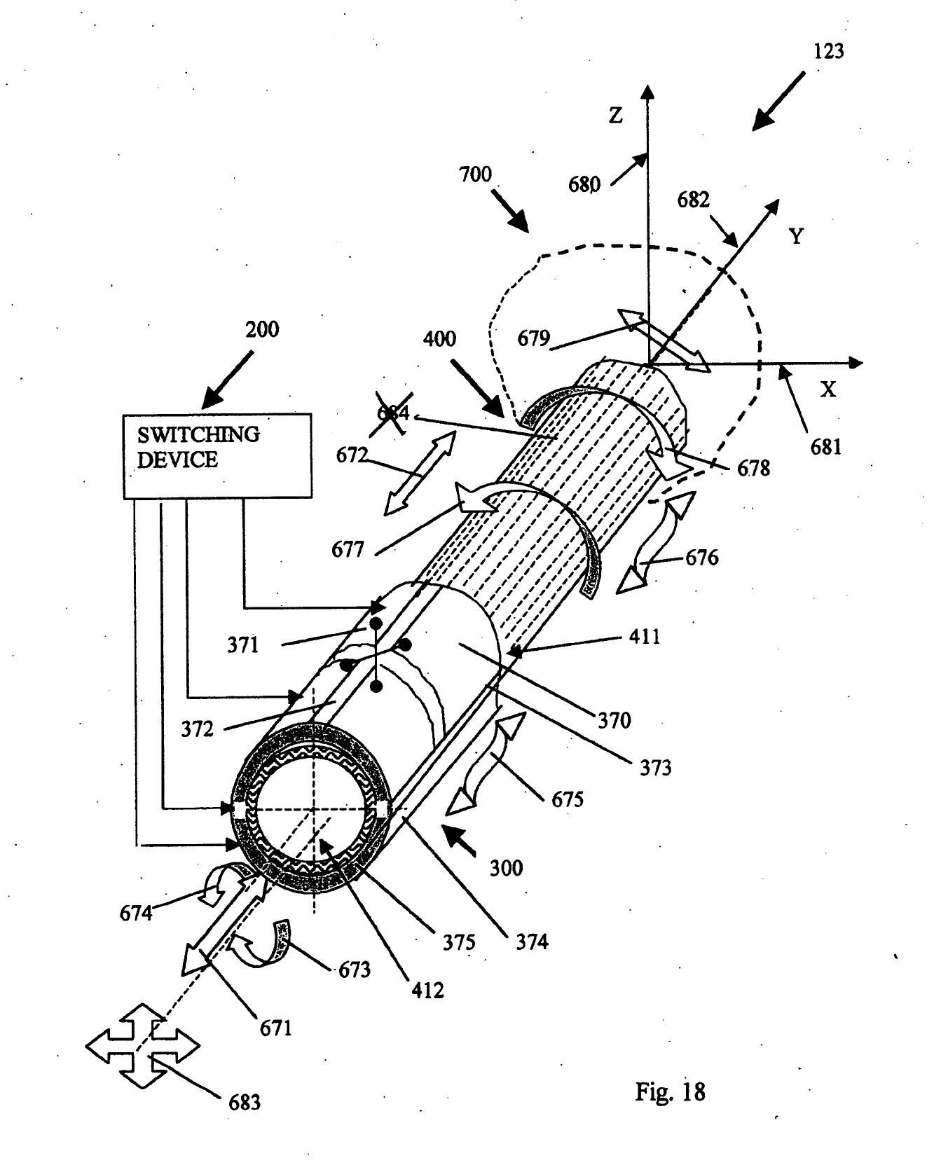

FIG. 18 illustrates a universal medical device undergoing the nanovibration coating process system 123 and involves the simultaneous application of different nanovibrations described hereinabove. Medical device 400 treated with the nanovibration coating process is attached to the body 700. Coordinates 680, 681 and 682 indicate the entrance of the medical device into the body. A piezo actuator of complicated configuration is attached to another side of the medical device. The actuator is capable of creating separately, simultaneously or in combination vibration modes of bending, longitudinal, torsional or any other character. The electrodes on the cylindrical piezo element 375 can be divided into different shapes 370, 371 and 374 (two or more electrodes), by means of electrically non-conductive material 372 and 373. By actuating the piezo element, controlled combinations of vibration modes can be created simultaneously and changed periodically. For example: longitudinal mode of piezo element 671 creates longitudinal vibration mode 672 in the medical device; bending mode of piezo element 672 creates bending mode 676 in the medical device; torsional mode of piezo element 673 creates torsional modes 677 and 678 in the medical device.

In such a manner, all vibration modes 671-683 may be achieved with one piezo element, applying different electrodes. The above-described effects may be achieved using separate sections of piezo element, which together form a cylindrical or other hollow shape. The above-described nanovibration coating may be achieved, as well, by placement of multiple piezo elements around the device, each of them having only one electrode.

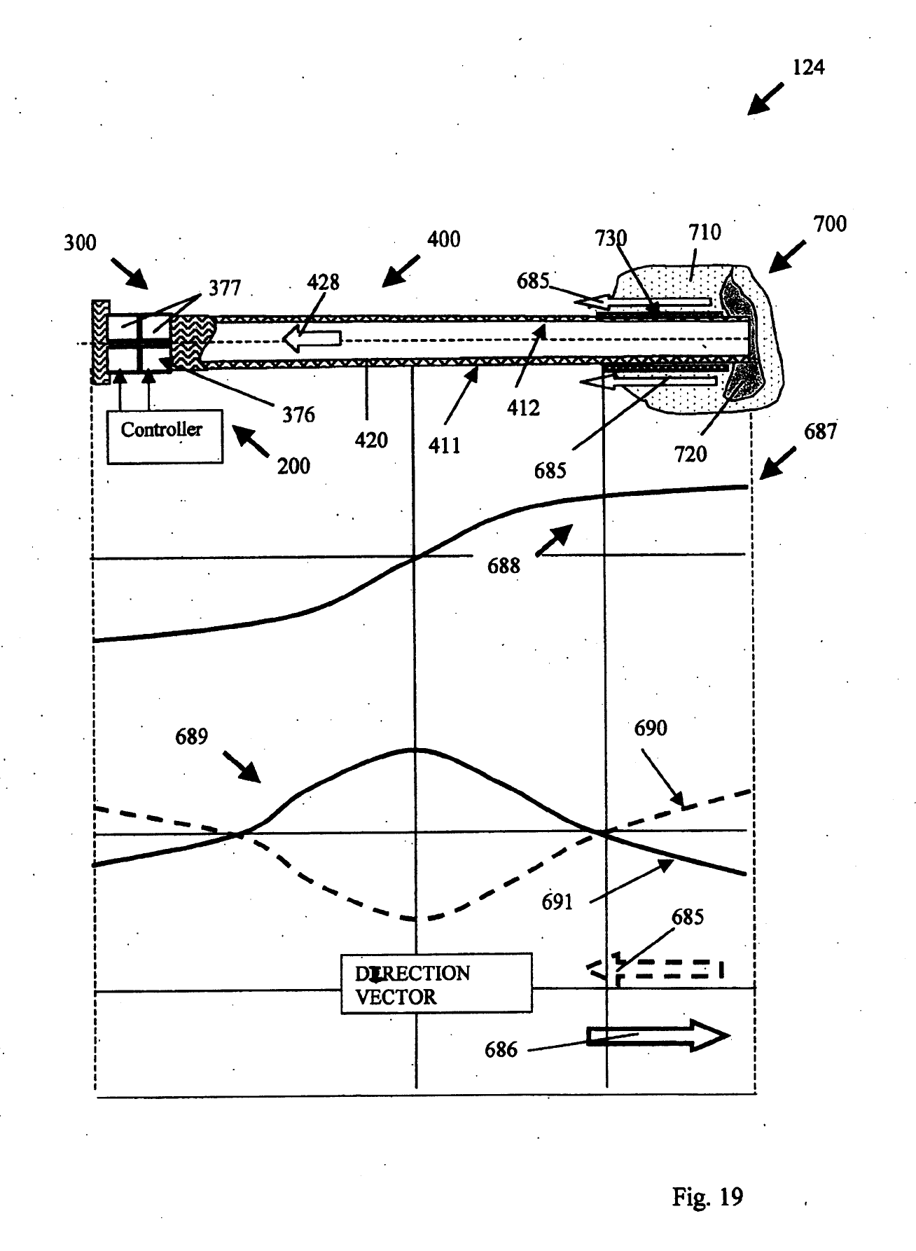

Another feature which may be achieved with the piezo element described above (in addition to a nanovibration coating) is to push or pull materials along said surfaces, including fluids and particulates suspended in them. The effect is shown in view 124 of FIG. 19. According to the illustrated embodiment, a specific combination of longitudinal and bending vibration modes is created, for example, the first mode of longitudinal vibration mode 688 and the second mode of bending vibration mode 689.

FIG. 19 shows medical device 400 to which is attached a cylindrical shape piezo element 376 with separated electrodes 377. On both external 411 and internal 412 surfaces of the medical device a nanovibration coating process is created. One end of the medical device 400 is introduced to the body at location 710, where it is necessary to pull out or push in the bacteriologic fluids 720. Surface biomaterials 730 are produced between external surface 411 of the medical device 400 and the body and it is necessary to pull them out of the body in direction 685. Piezo element 376 achieves this removal effect by simultaneously creating a first mode of longitudinal vibration 688 and a second mode of bending vibration 689. The surface points of the end of the medical device are excited with the above-described vibration modes. When the bending vibration mode phases coincide with longitudinal vibration phases, the movement in direction 685 is produced at the end of the medical device; when these phases do not coincide, and bending vibration mode 691 is 180° opposite in phase to longitudinal vibrations, movement is produced in the direction 686.

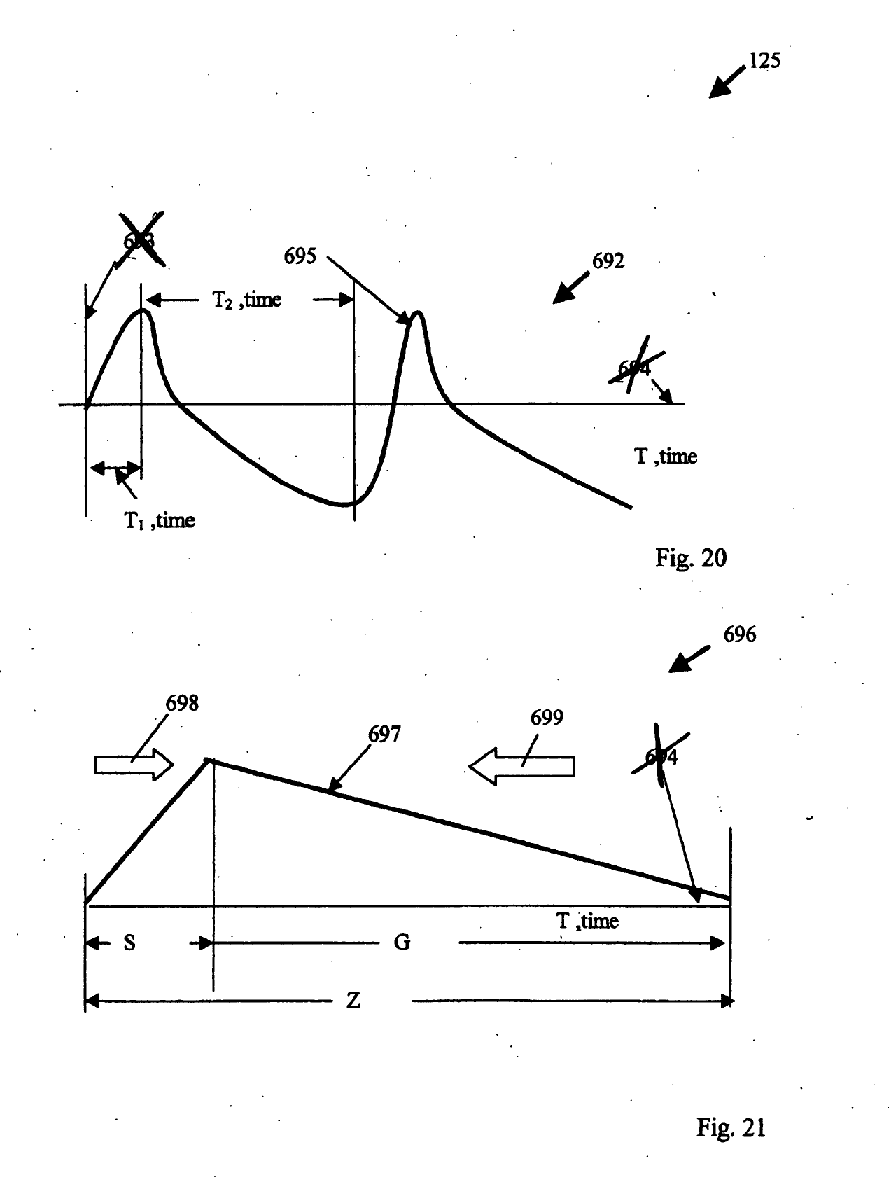

FIG. 20 and 21 present schematic diagrams of flow acceleration 692 and movement 696 illustrating a second way to achieve the pushing or pulling of materials, including fluids and particulates, along said surfaces, in addition to applying a nanovibration coating effect. This method for pushing or pulling biological matter is better when the internal channel of the medical device is used.

In FIGS. 20 and 21, the coordinate 693 in the diagram matches acceleration 692 and deceleration 695 of the movement of the medical device end points along a time scale. The first period T₁=S is considerably shorter relative to the second period T₂=G. Consequently, in the forward direction medical device end points 698 are moving quickly and in the backwards direction 699 are moving slowly. Graph of movement 696 shows that the vibration process has an effect on pushing out biological matter through an internal channel of the medical device. Contrary, if it is necessary to pull in biological matter, the G/Z must be considerably shorter than S/Z. In addition, the direction of the movements can be simultaneously opposite one to another on each opposing surface in result of manipulating with the first and second described above methods in one device (such device is shown in the FIG. 19).

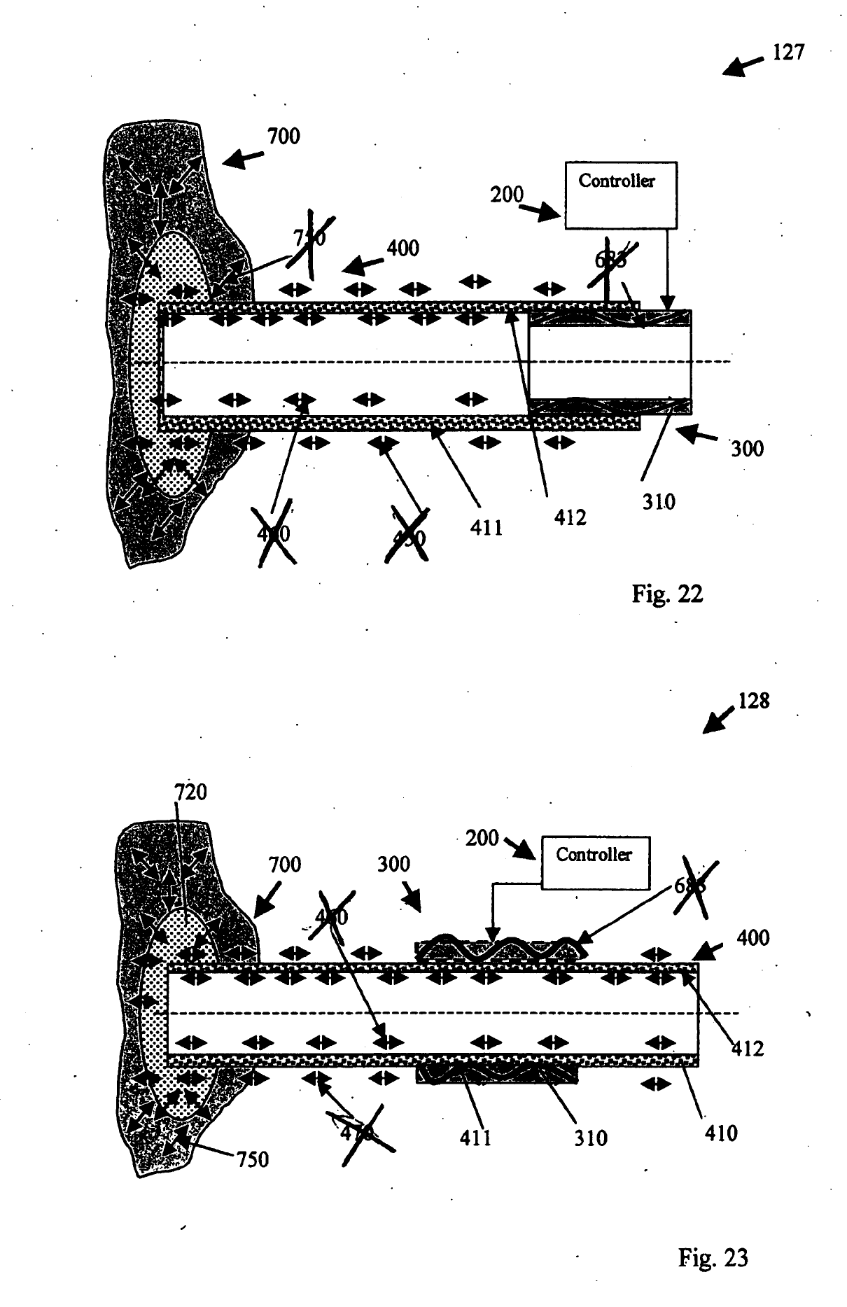

Several methods for attaching the mechanical actuators to medical devices for creation of a nanovibration coating process on their external and internal surfaces are shown in FIGS. 22, 23, 24 and 25.

FIG. 22 shows the scheme 127 illustrating attachment of a mechanical vibration device to a standard medical device, wherein the external surface of the vibration device is attached to an internal surface of the medical device. Vibrating piezo element 300 is attached to the internal surface 412 of the medical device, in order to create a stronger nanovibration coating process on the internal surface of the medical device. Nanovibrations are not transmitted through any walls themselves forming the medical device but rather propagate along exterior surfaces.

FIG. 23 shows the scheme 128, illustrating attachment of a mechanical vibration device to a standard hollow medical device such as a catheter, wherein the internal surface of the vibration device is attached to an external surface of the medical device. In this case, the nanovibration coating process will be stronger on the external surface, and will prevent biofilm formation to the external surface of the medical device.

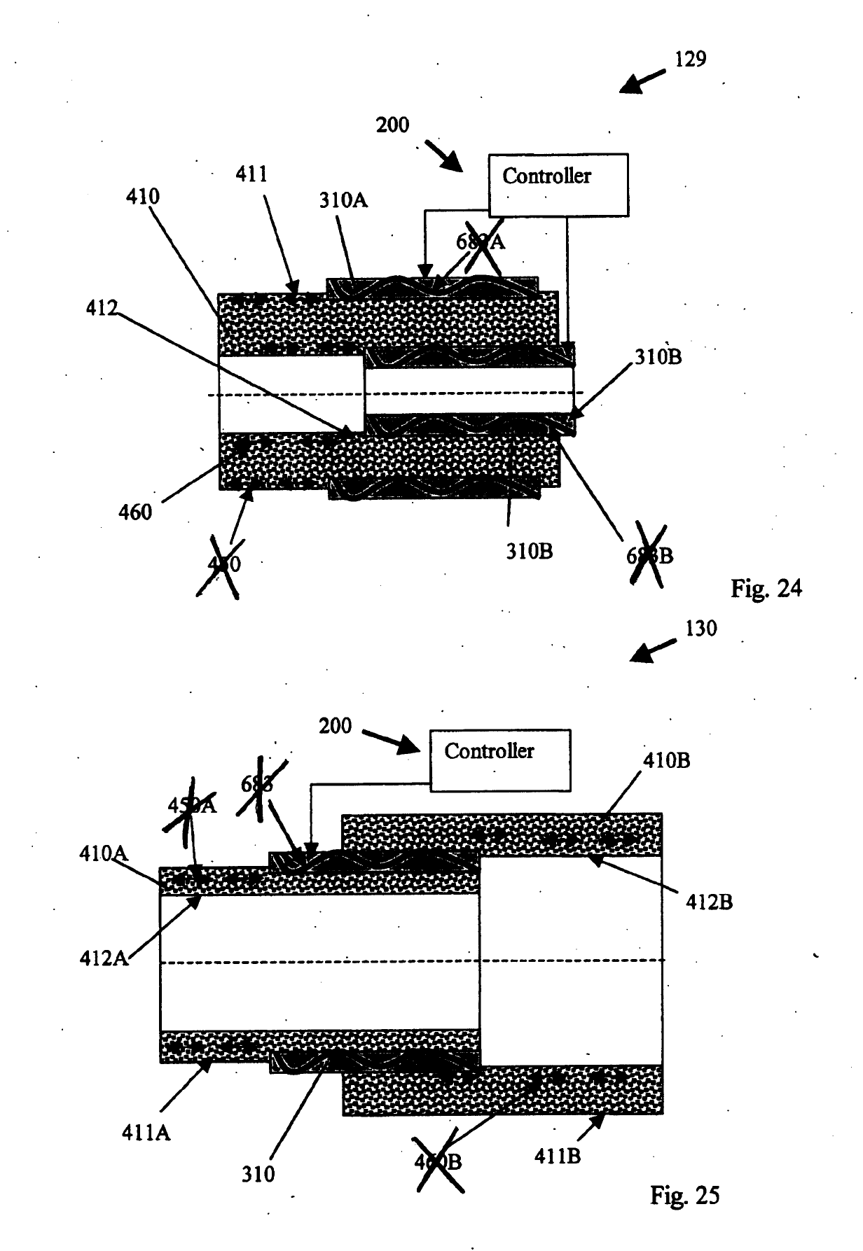

FIG. 24 shows the scheme 129 wherein two pieces of vibration devices can be applied—one internally and another externally. When two cylindrical piezo elements 310A and 310B are attached to one medical device, it is possible to create different nanovibration coating processes on external 411 and internal 412 surfaces of the same device 400.

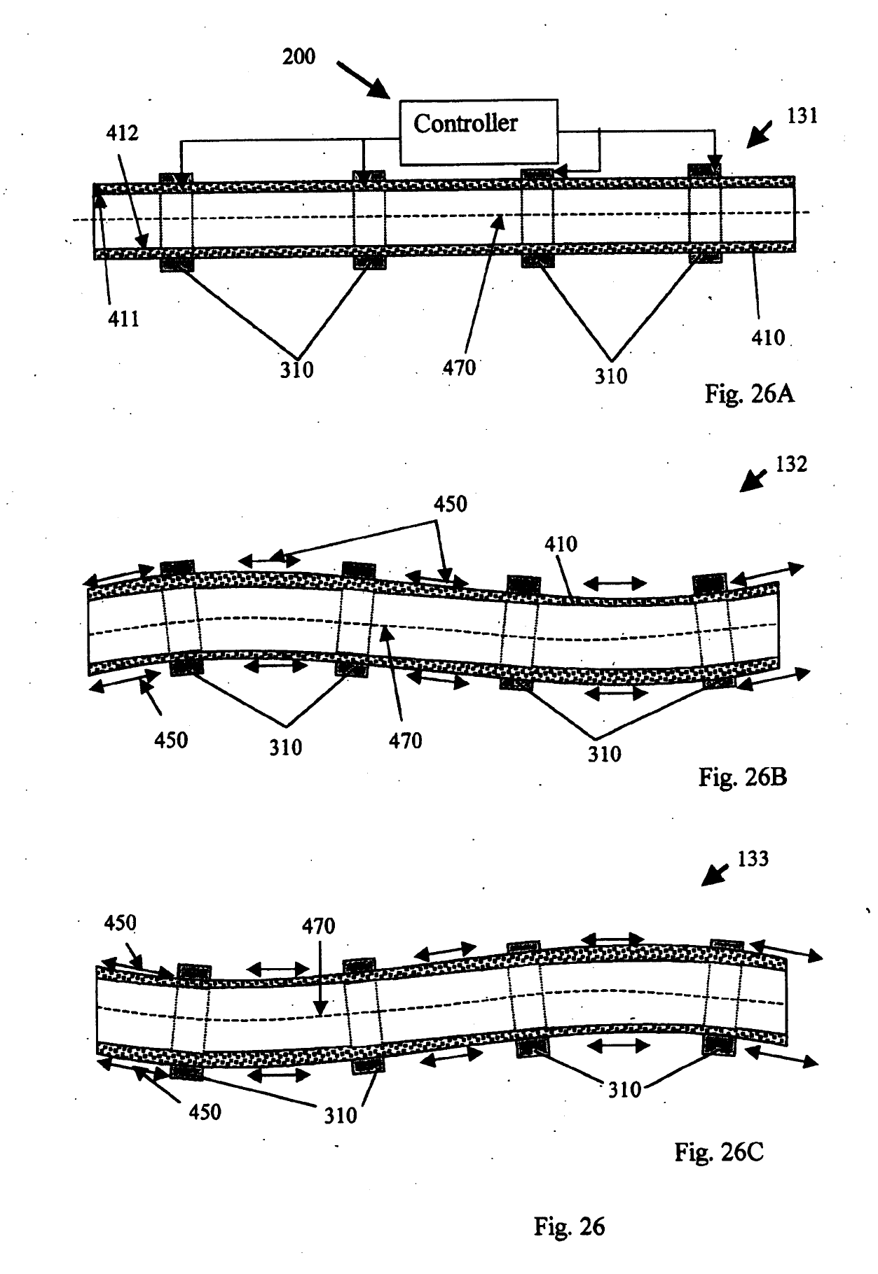

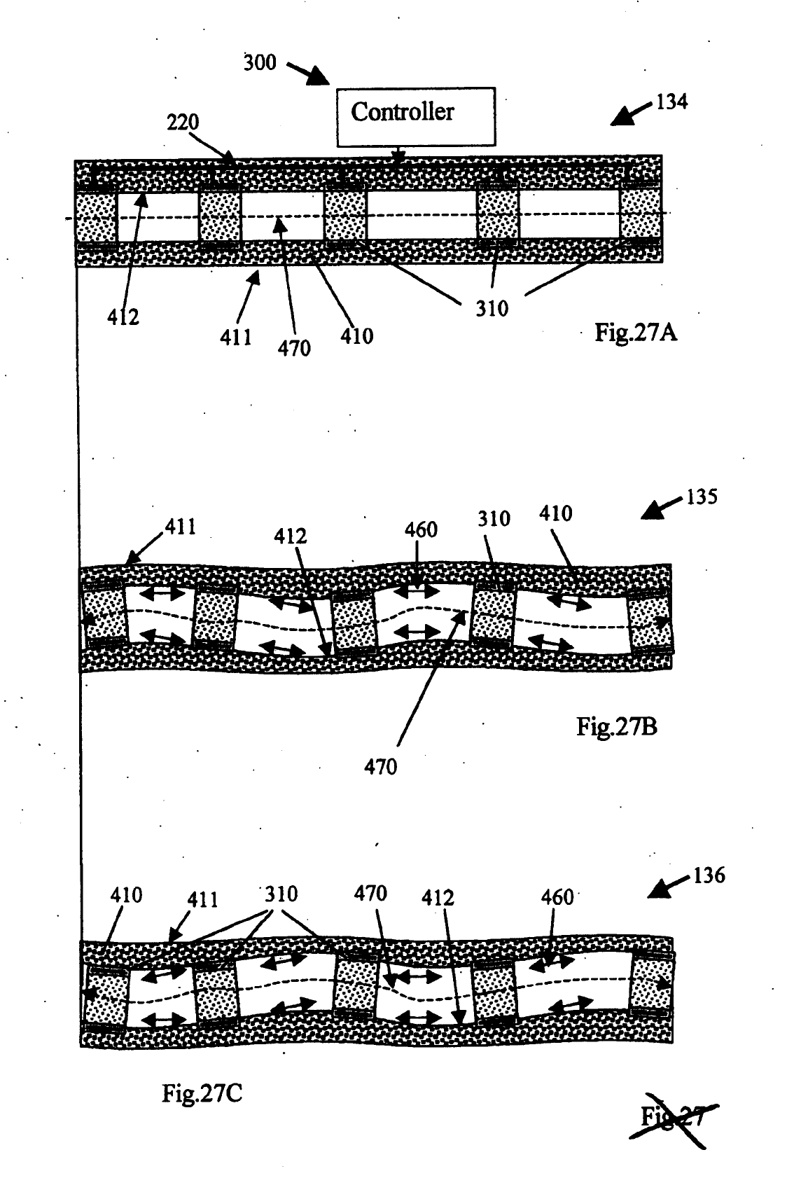

FIG. 25 shows the scheme 130 wherein the medical device has a one piece cylindrically shaped piezo element 310, and external and internal surfaces are attached to two different hollow medical devices 410A and 410B. Elongated medical devices may require multi elements to achieve the desired effect (each element working as described above). If these vibration elements are working in the same phase of an electrical signal, the vibration character (FIG. 26A) repeats sequentially. Where these vibration elements are working in different phases of electrical signal, as in FIG. 26B and FIG. 26C, the process is more complicated. When the medical device is furnished (attached) with actuators having two or more characteristic signals, the following is achieved: the wall all along a length of the device vibrates simultaneously in natural vibration of longitudinal, bending and torsional modes. The effect can be achieved by either attaching the actuators internally or externally to the medical device surface. Attachment of elements externally as in FIG. 26 creates a stronger vibrating effect on the external surface. A reverse situation where the attachment is internally results in a stronger internal vibration coating as shown in FIGS. 27A-C.

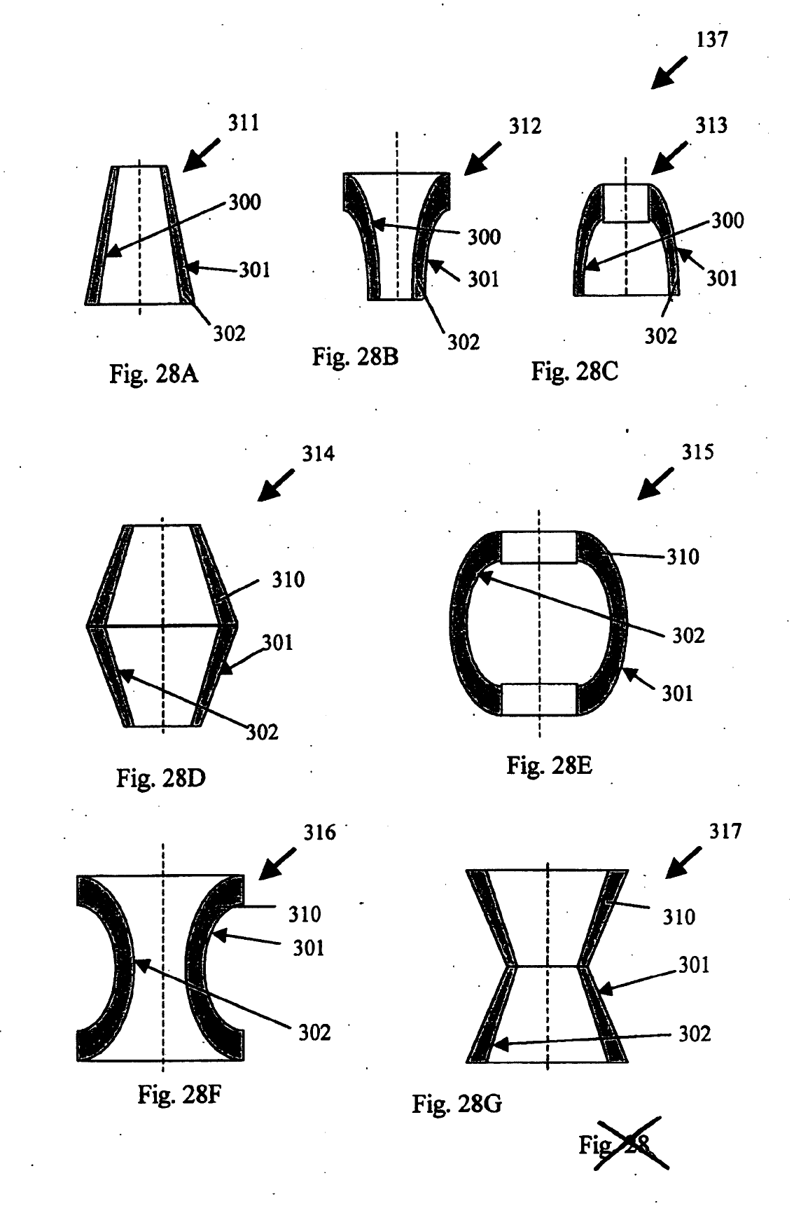

FIGS. 28A-C shows the view 137, which illustrates a variety of shapes of piezo elements for generation of a nanovibration coating process and preventing biofilm formation on the surfaces of medical devices. These shapes can be selected from convex, concave and tapered arrangements (views 311, 312, 313). At least two shapes of the group consisting of convex, concave and tapered can be used for designing piezo elements (as is shown in FIG. 28D-FIG. 28G).

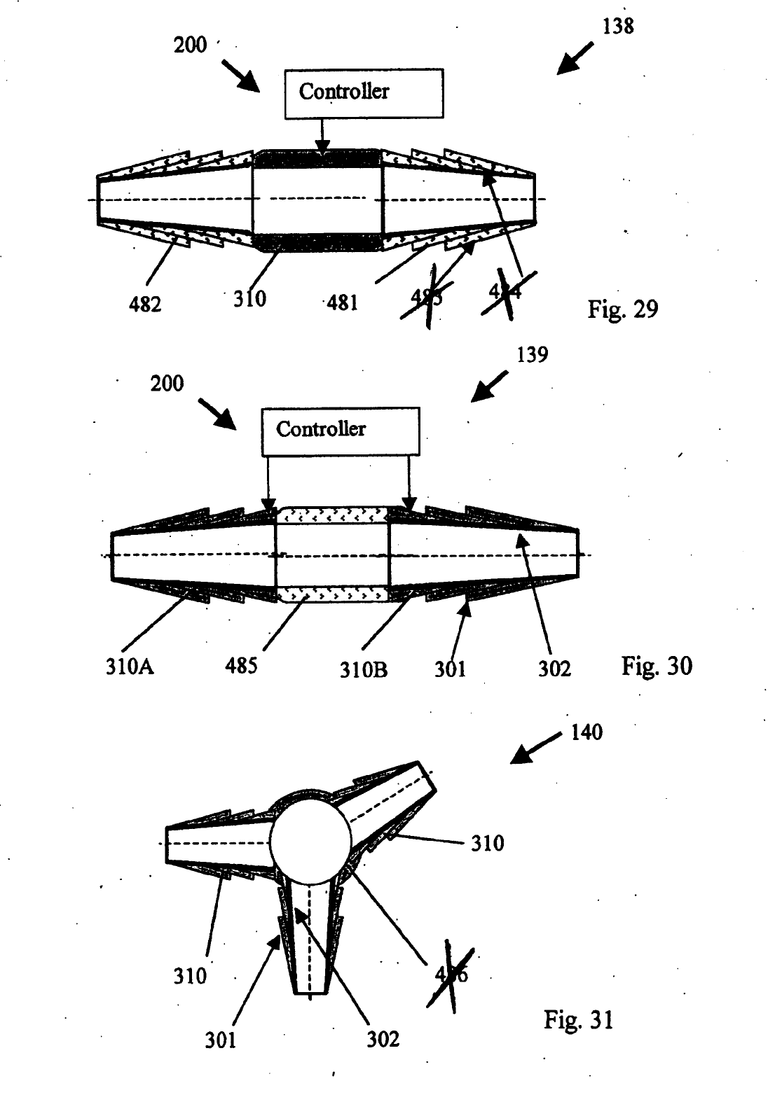

FIGS. 29 through 31 illustrate piezo actuator shapes with serrated outer shapes useful to ensure good connections to a medical device. FIG. 29 illustrates a case wherein one cylindrical piezo element 310 has two plastic connectors 481 and 482. FIG. 30 is the view 140 of two piezo elements 310A and 310B connected between them by means of plastic tube 485. FIG. 31 illustrates a piezo actuator-connector consisting of three piezo elements 310 which create nanovibrations on external and internal surfaces and prevent formation of biofilm.

According to these embodiments, an actuator is connected on one or more sides to an intermediate material having physical mechanical properties that modify the original coating process. In this way, one can control the coating effect through the change of the material of the connecting elements. The same can be achieved by connecting to one actuator more than one connector having different physical mechanical properties. The result is that multiple nanovibration coatings are achieved on different sections of the device, depending on the physical mechanical properties of the connector.

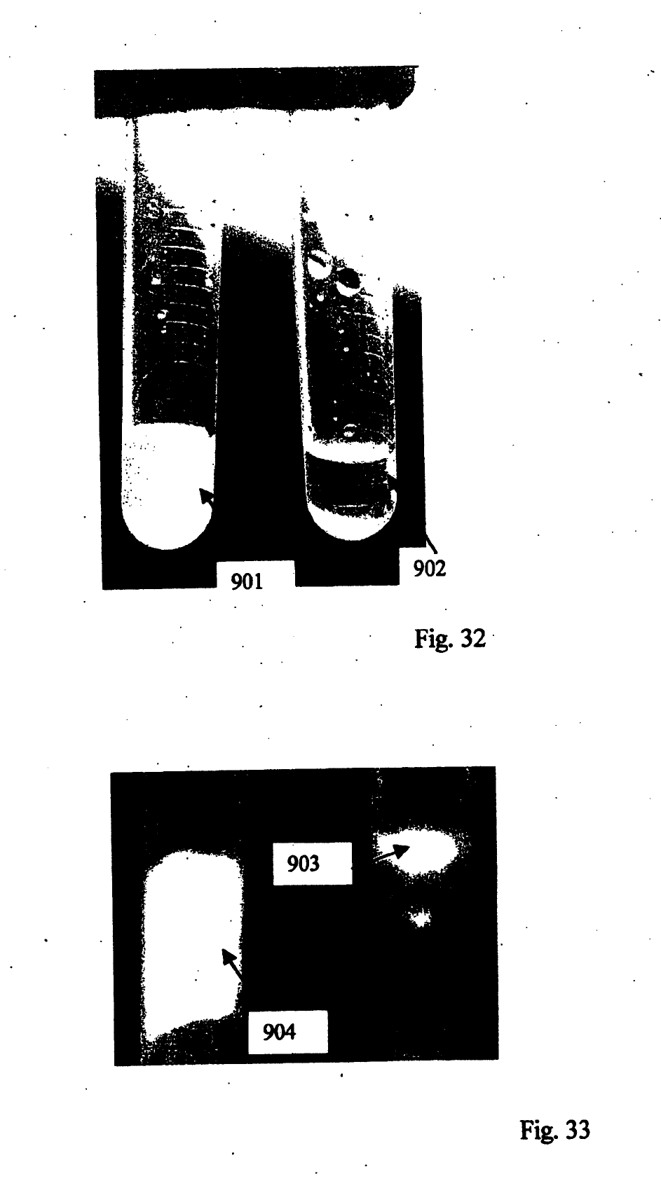

FIGS. 32-37 describe results of conducted experiments designed to prove feasibility of the present process to prevent biofilm formation in various media. The experiments described hereby follow our observations that biofilms do not develop on and near vibrating surfaces. Our observations show the establishment of biofilms requires attachment to hard surfaces. The vibrating surfaces are not perceived by the bacteria as a solid surface on which it can establish a biofilm.

FIG. 32 shows the experiment related to the medical device with nanovibration coating process for preventing the development of biofilms in fluid media. After 21 days, the fluid in the control dish without a nanovibration coating process started to change color and gradually a whitish biofilm formed. After 21 days, the liquid from each flask was sampled, vortexed and placed into a test tube. The liquid 902 from the flask containing the medical element with nanovibration coating process remained clear. The fluid 901 from the control (without nanovibration coating process) was of milky appearance and loaded with bacterial clumps, and biofilm fragments were confirmed by microscopic examination.

FIG. 33 shows the experiment related to the medical device with nanovibration coating process for preventing biofilm formation on flexible media, such as latex. Nanovibration coating process in the medical device was generated at a distance and propagated through a tube of 50 cm length. A latex tube was filled with non sterile spring water (a glass of water standing at room temperature for several days). Two tubes were used—with vibration and as control. Each tube was filled with 7 cc of the above spring water. On termination after 25 days, the water was drawn by a syringe from the tubes and centrifuged at 6000 RPM for 5 minutes. The pellets were resuspended in 250 ml water each—and introduced into hemocell capillary glass tubes and centrifuged at 12000 RPM for 5 minutes. Biofilm matter from the control tube (without nanovibration) is shown as 904. Biofilm matter from the tube of the medical device with nanovibration coating process is shown as 903. These results constitute a further proof that this technology effectively prevents biofilms formation in liquids in a closed system made of flexible material (e.g., a catheter tube). The vibrations were generated from one point and uniformly propagated through the flexible material grade latex tube. On macroscopic inspection of the vibrated tube there were no foci of biofilms to be seen.

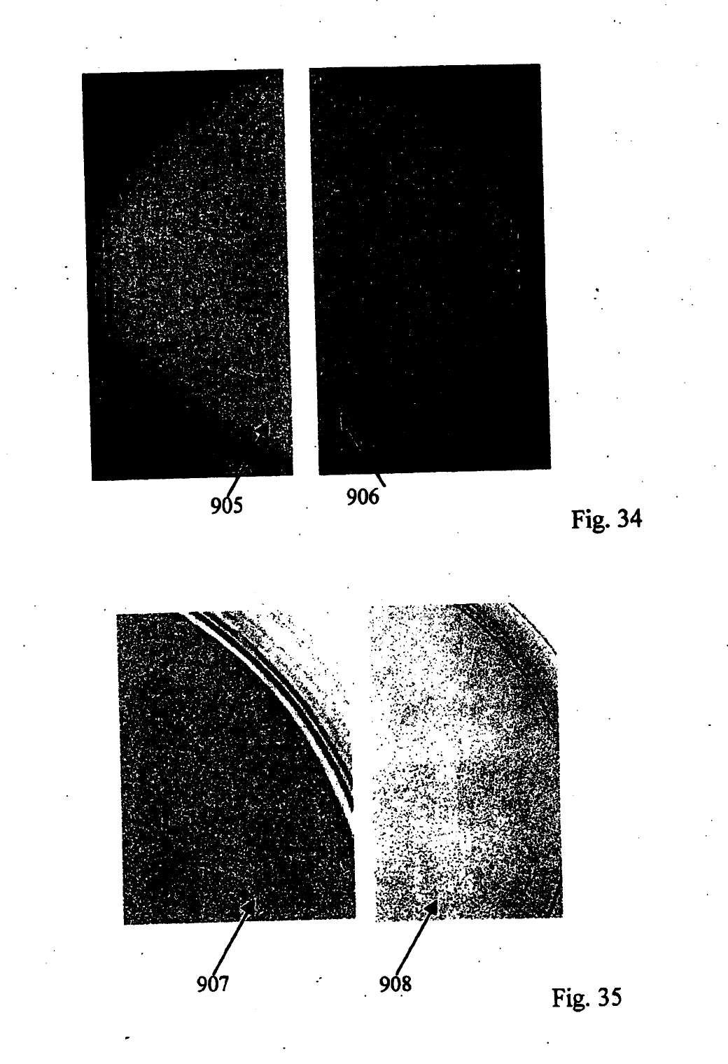

FIG. 34 shows the experiment which relates to the medical device with nanovibration coating process on solid ceramic surface, coated with a flexible media of thin silicone layer. Inspection revealed a gradual development of biofilm on the solid ceramic surface coated with a flexible media of silicone layer, which was without nanovibration coating process, as seen in 906 (on day 12). By contrast, as seen in 905, biofilm did not develop on the solid ceramic surface coated with a flexible silicone layer subjected to a nanovibration coating process. These experiments clearly indicate that a nanovibration coating of the elements inhibits bacterial adhesion and attachment directly to the treated surface and even to a considerable distance from the treatment. The surfaces remained smooth in all experiments as biofilms did not develop. However, biofilms did form on surfaces without a nanovibration coating treatment and the biofilm was intimately and tenaciously attached to the surface.

FIG. 35 shows the experiment related to a medical device with nanovibration surface coating process for preventing of biofilm formation in semisolid media. The results show that the fluid from the device with nanovibration coating surface 908 remained clear compared to the control device, without nanovibration coating surface 907. After removal of the fluid from dishes, the agar from each dish was inspected for turbidity: the dish without nanovibration element is seen to be heavily loaded with bacteria embedded in the semisolid substrate (agar—agar). The agar in the dish with nanovibration treatment remained clear. The differences shown in FIG. 35 were taken after the dishes were drained from liquid media. While in the control dish 907, a heavy growth of bacteria is seen, the medium removed from the dish with nanovibration element 908 was clear and devoid of bacterial growth (PROTEUS MIRIBILIS). This growth causes the media to change color, as in 907, because of a heavy presence of microorganisms embedded therein. Nanovibration surface coating process has the potential of preventing bacterial biofilm formation and bacterial growth in semisolid media.

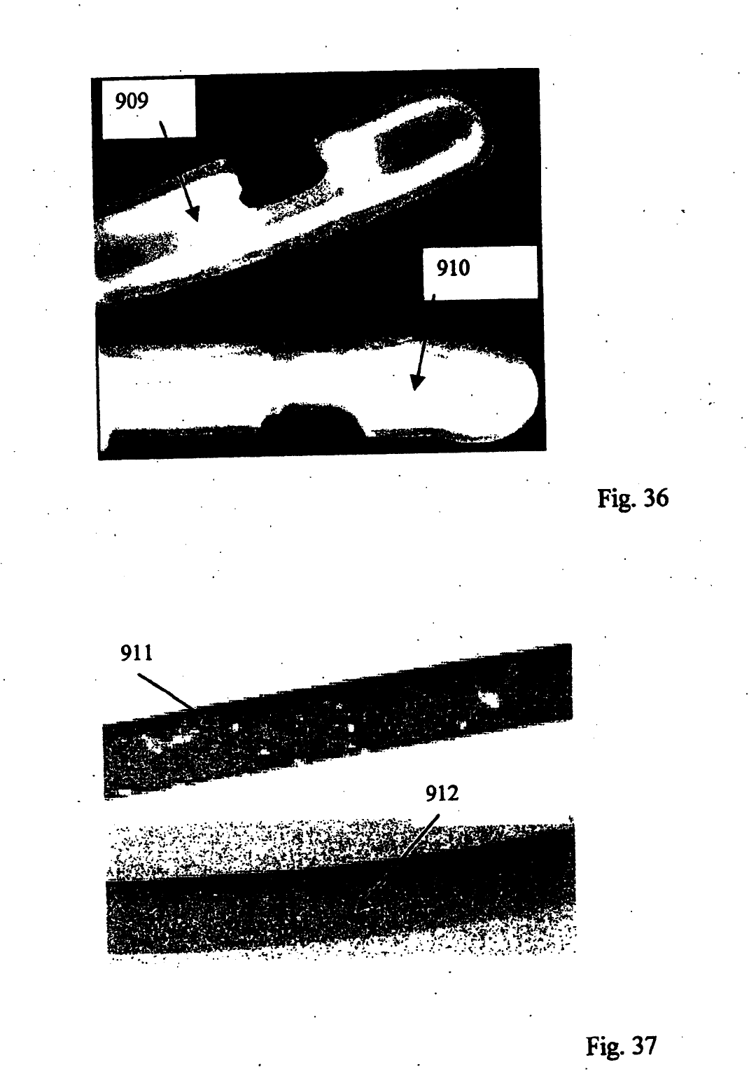

FIGS. 36-37 show the experiment related to a latex medical device in which biofilm formation was prevented on internal and external surfaces of the tubes that were vibrated. Consequently, it is evident that the nanovibration surface coating process inhibits the growth of biofilm on a latex surface. Moreover, as seen in 911, crystals are precipitated on the latex tube and the precipitation is inhibited on the nanovibration surface 912. Propagation of a nanovibration surface coating process along an entire 4 meter length of medical grade latex tube effectively prevents biofilm formation. The experiments further demonstrate the efficacy of nanovibration surface coating process to inhibit biofilm with dosed targeted and uniform energy transmission through diverse media. FIG. 36 shows the experiment related to treatment of internal latex media of a medical device with nanovibration surface coating process for preventing biofilm formation. The FIG. 36 photo reveals improvement on the internal activated surface on the latex media 910 relative to the internal latex media 909 without nanovibration treatment.

The results of the nanovibration surface coating process effectiveness in preventing biofilm formation is shown in Table 1. Levels of biofilm and incrustation/crystals are represented by (−) indicating low or absent amounts and (+) each indicating higher amounts.

In these in-vitro experiments, nanovibration surface coating process was achieved by generation of elastic waves in the frequency range from 28 KHz to 5.5 MHz, and amplitudes of about 5-20 nanometers.

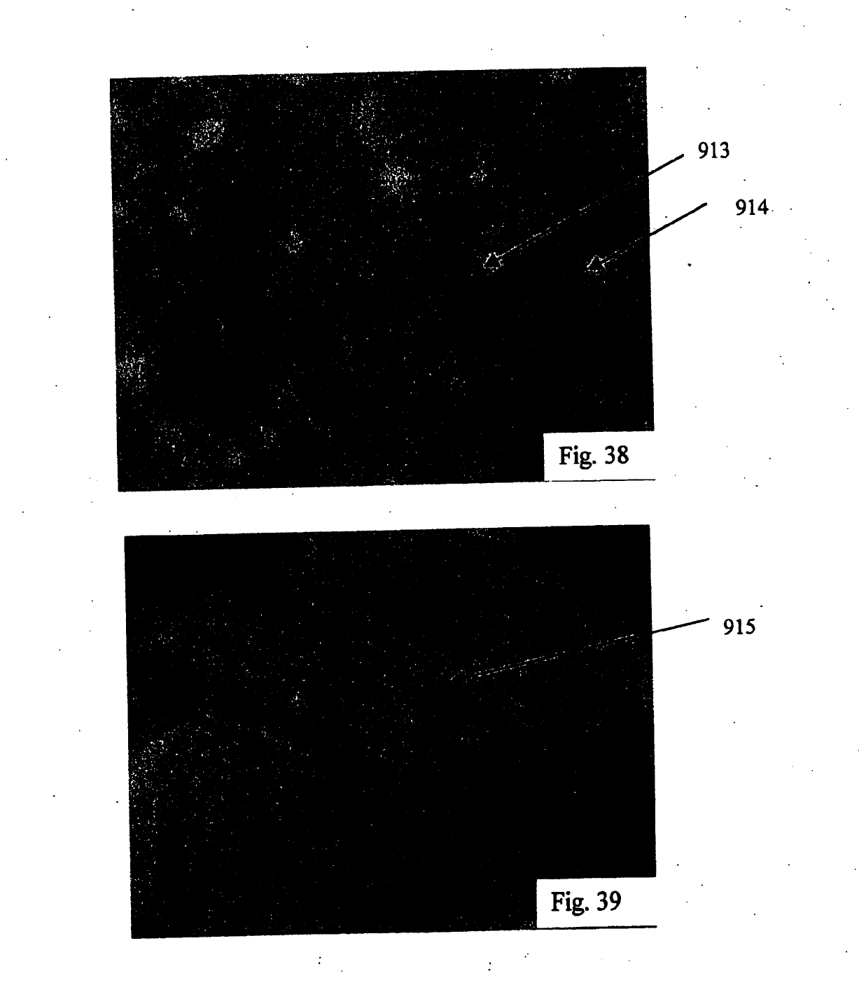

The results of in-vitro experiments of nanovibration surface coating process in urinary catheters (UnoPlast Company) are shown in FIG. 38 and FIG. 39. FIG. 38 shows a scanning electron micrograph (SEM) image of a urinary tract of an animal subjected to a non-activated catheter. After 3 days, the tissue is destroyed. The dead cells are shown as areas 913 and 914. FIG. 39 shows a scanning electron micrograph (SEM) image of epithelium from a urinary tract of an animal subjected to an activated catheter. After 8 days, the cells are intact, shown as areas 915. Infection was prevented using nanovibration coating process of the external and internal urinary catheter surfaces.

The applications described here below illustrate the variety of cases where the problem of preventing biofilm formation is important.

An additional common application is used with a urinary catheter. Herein, the actuator can be placed on a connector, on a part of the catheter outside the urinary tract, on a urinary bag separately or on all of them together for the purpose of biofilm and incrustation prevention.

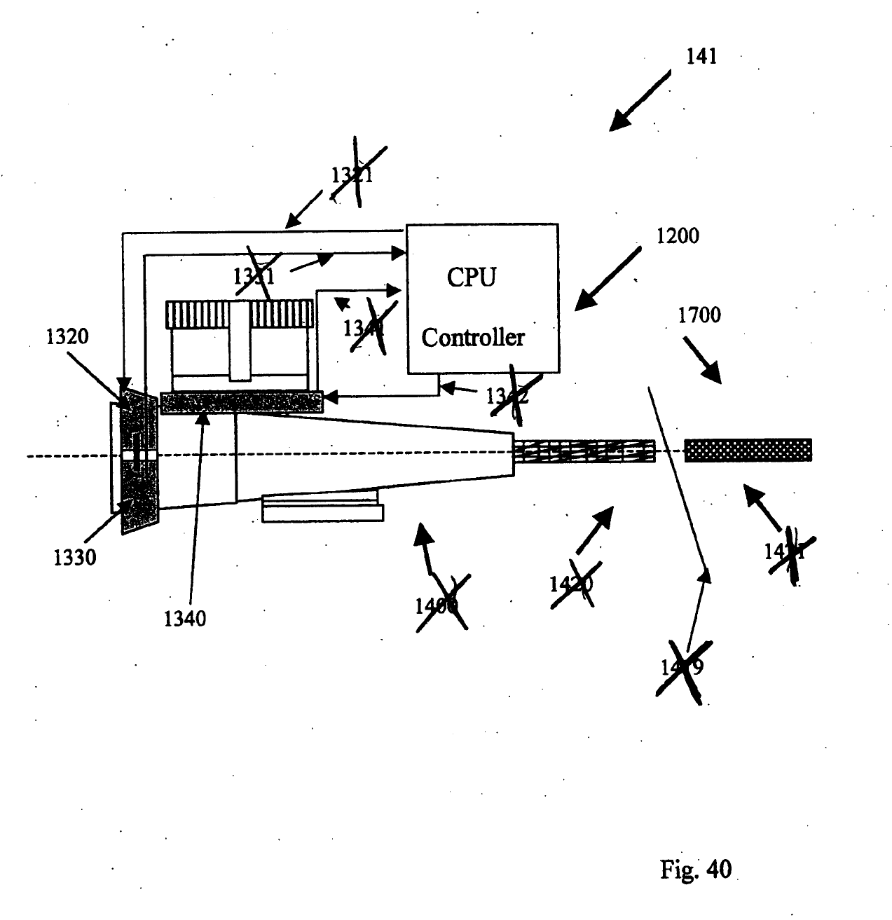

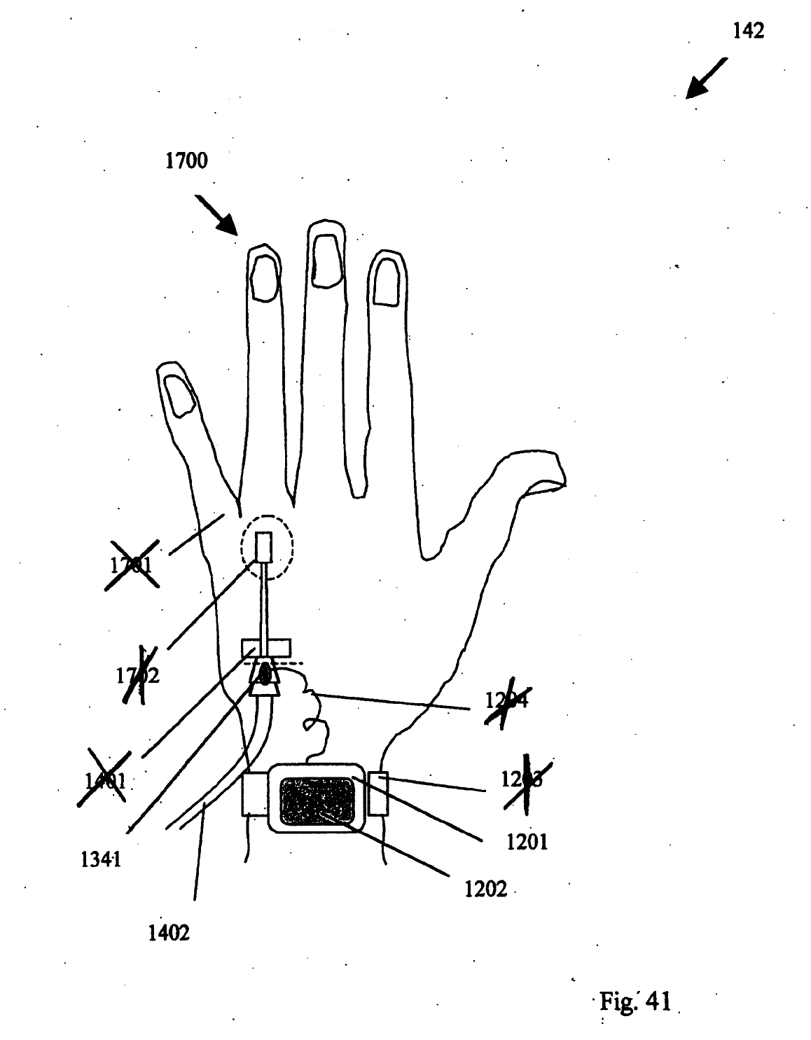

In FIG. 40 is illustrated a standard peripheral intravenous catheter system. Herein, at least one piezo element 1400 is attached to a hub of the catheter. The piezo element includes a sensor 1320 and an actuator 1330 for nanovibration coating. The piezo elements are excited by signals from CPU 1200 through a variety of controls and signal receivers from the sensor. FIG. 41 in view 142 illustrates how the system of FIG. 40 can be attached to a human hand 1700 locating thereon the CPU and energy source units 1201, 1202. Indeed, the actuator 1341 can be placed on any part of the medical device 1402 including, but not limited to, fluid reservoirs, pumps or any ancillary equipment. One or more actuators can be placed at different points along the system, especially those points more susceptible to the entry of microorganisms.

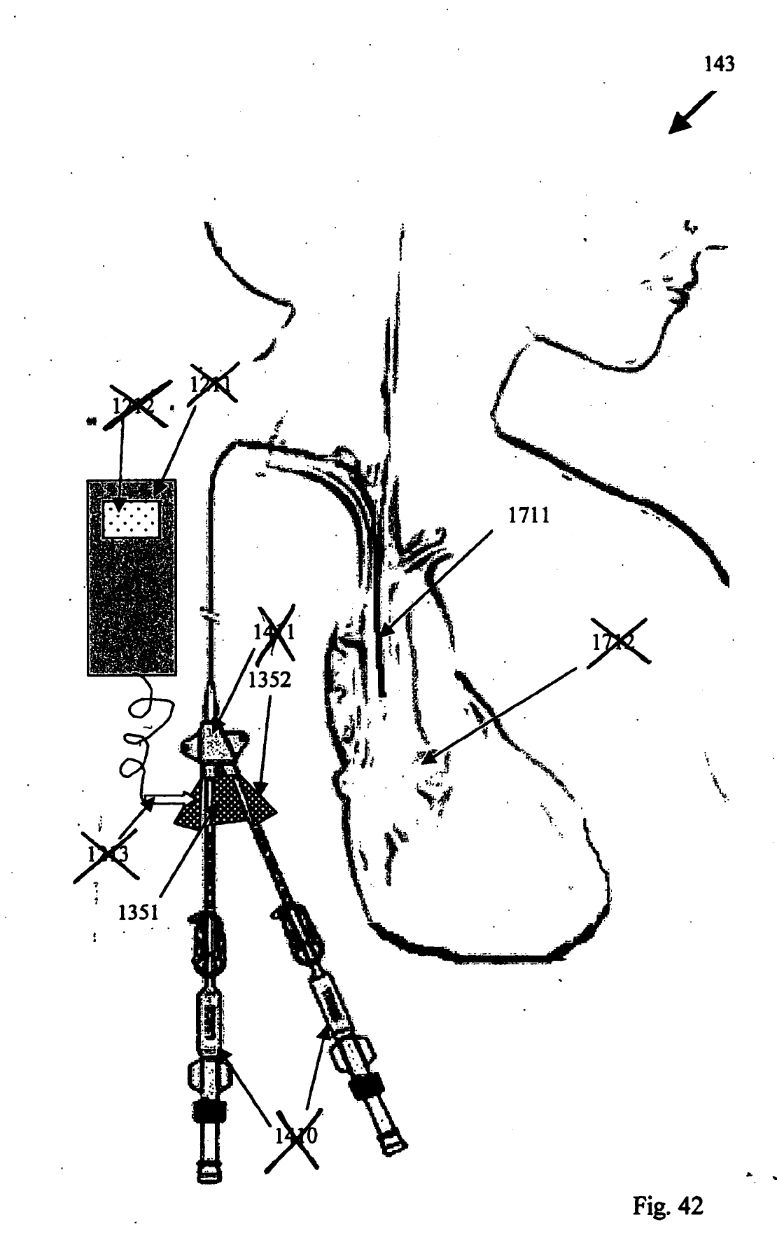

Nanovibration coating actuators can be attached adhesively to any standard medical device. FIG. 42 in view 143 illustrates a mode of attachment. More particularly, the piezo element is attached to adhesive pad 1351, 1352 on the convergence of all the catheter elements or on each of them separately. The actuator can also be placed on a part of the line.

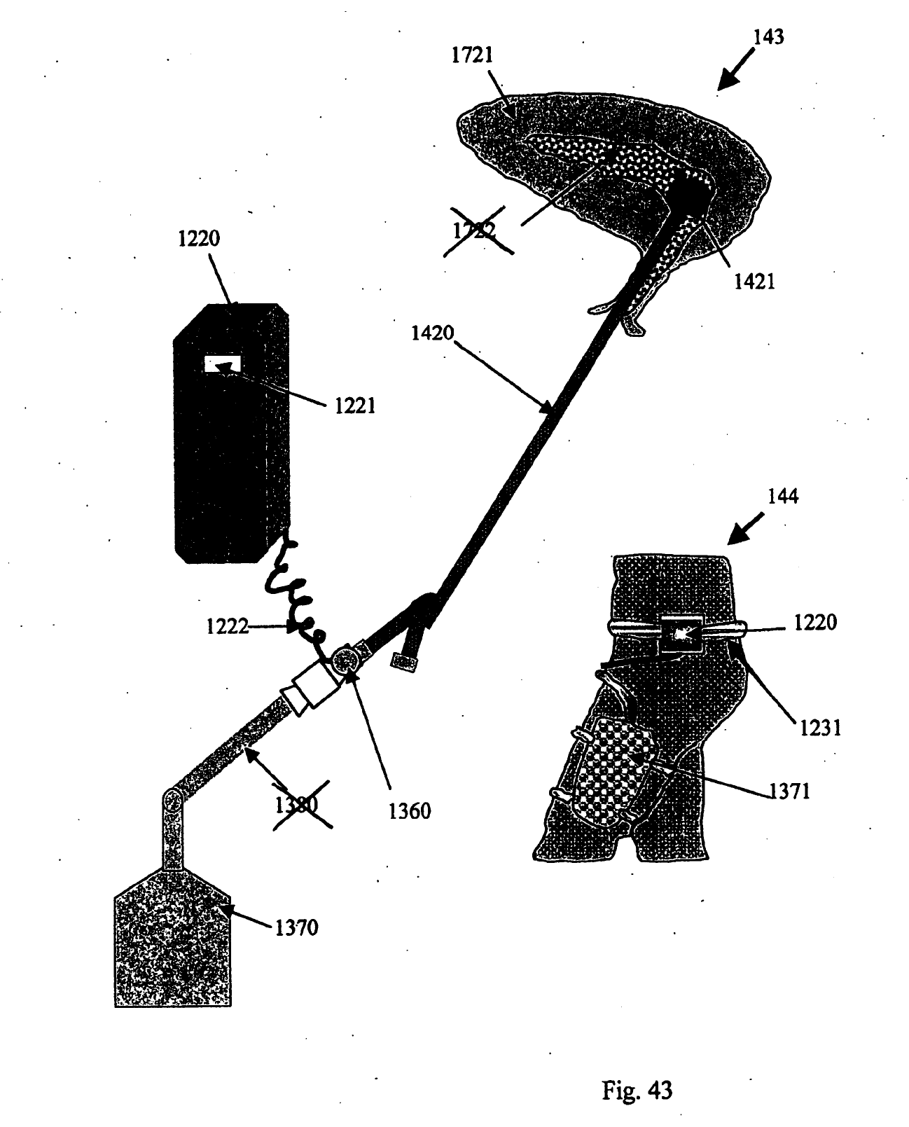

Another common possible application is with a urinary catheter. Here, the actuator can be placed on the connector, on the bag, on a portion of the catheter outside the urinary tract, on a urinary bag separately or on all the aforementioned components for the purpose of biofilm and encrustation prevention. FIG. 43 is particularly illustrative of these options.

FIG. 43 illustrates options 143 and 144. Urinary catheter 1420 is connected with piezo element 1360, which is connected respectively with CPU 1220, having display 1221 by a wire 1222. Catheter 1420 and balloon 1421 is inserted into the urinary tract 1721 and as a result of created vibrations, biofilm formation is prevented on all surfaces of the catheter. In one embodiment of the present invention, the piezo element may be attached only on the urinary bag 1370. In this arrangement, the excited vibrations will avoid formation of biofilm not only on the surfaces of the urinary bag, but also on the surfaces of catheter.

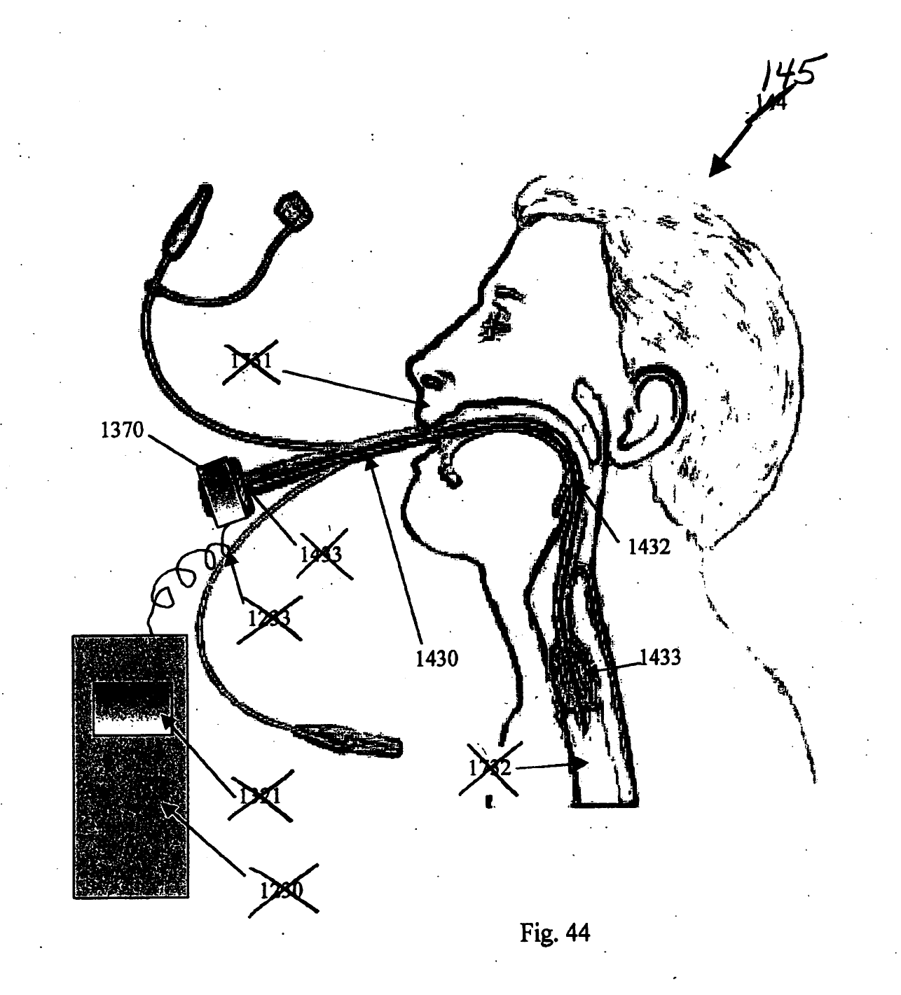

FIG. 43 with view 144 explains additional possible variations. A urinary bag 1371 can be attached to the leg of the patient, while CPU 1220 and the battery are attached to the belt 1231. FIG. 44 shows another embodiment in view 145 depicting an endothracheal ventilation tube (which is a major cause of death due to pneumonia, resulting from biofilms). Tube 1430 is vibrated at crucial points 1432, particularly around balloon 1433. The actuator 1370 can be placed on any part of the line connected to the system. One or more actuators can be attached or directed against any point serving as entry point for microorganisms. Ventilation machines as the one illustrated in FIG. 44 are at high risk to become contaminated in standard practice. Our technology enables prevention of biofilm formation at any part of the system, which can be furnished with actuators. Body tissues which are in contact with the activated medical device are protected. In this way, arteries, veins, mucosal membranes and other organs and body cavities are protected from colonization with bacteria and formation of biofilms.

All the aforementioned descriptions and embodiments are not to be considered as restricted to use in standard medical devices. It will be clear to those skilled in the art that the nano vibration coating process of the present invention can be incorporated or embedded or integrated with any future design medical device or accessories.

1. A method for preventing biofilm formation associated with indwelling medical devices, the method comprising forming a nanovibration coating process over surfaces of medical device, by communicating mechanical vibration energy to the medical device to enhibit entry of micro organisms from external and internal areas of the medical device.

2. Apparatus for preventing biofilm formation associated with indwelling medical device, the apparatus operative to generate a nanovibration coating process over the medical device surfaces, by generating electric signals by a processor and transforming the electric signals to mechanical waves with nano amplitudes, and transmitting the mechanical vibrations by means of traveling waves to the medical device.

3. The apparatus of claim 2 comprising ability to form nanovibration coating process on external, internal, torsion surfaces and their binding lines of medical device—simultaneously or separately, by means of applying mechanical vibration energy to the medical device.

4. The apparatus of claim 2 comprising ability to excite nanovibration coating process all over medical device surfaces, by applying mechanical vibration energy to the device using periodical, non periodical, electromechanical, electro-magnetic energy sources.

5. The apparatus according to claim 2 wherein the nanovibration coating process has a spectrum plot ranging from about 0.001 to 10 MHz

6. The apparatus according to claim 2, wherein the nanovibration coating process have amplitudes ranging from about 1 to about 50 nanometers.

7. The apparatus according to claim 3, comprising piezo element, which is adjusted to be in resonance of the system, consisting of piezo element attached to standard medical device, for optimal process.

8. The apparatus according to claim 2, whereas controller comprises: power supply (battery or other existing power supply), central processing units with memory nanovibration oscillator for pulsed or harmonic signals.

9. The apparatus according to claim 2, comprising controller to achieve the system resonance, which depends on piezo element attachment place, attachment type and the surrounding liquid (temperature, physical characteristics, quantity).

10. The apparatus according to claim 8 comprising modulators and switching device of vibration methods, which transmits electrical signal to mechanical vibration device for exciting complex of mechanical vibrations to excite nanovibration coating process on standard medical devices, in relation to patient health status and the program of medical personal to adjust and match biological cycles, changes in body temperature pathological conditions.

11. The apparatus according to claim 8, comprising: nanovibration oscillator (with range of frequency from 11 Hz to 50 MHz), two switching devices, which switch together or separately frequency and amplitude modulators (using cycling ring and additive synthesis modulators).

12. The apparatus according to claim 8, comprising the second switching device, which chooses and amplifies vibration mode of the mechanical vibration actuator, using single phase, two phases and multi phase electrical signal.

13. The apparatus according to claim 8, comprising: receiver device for information on nanovibration process and audio, video, alarm system to inform the status of nanovibration process in standard medical device.

14. The apparatus according to claim 3, with different amplitude and frequency, ranges of nanovibration coating process created using the first harmonics of vibration modes applied separately (of longitudinal, bending, torsion, or other type), proceeding to nanovibration coating process in the range of up to 0,5 Hz.

15. The apparatus according to claim 3, comprising ability to combine simultaneously two vibration modes and effecting in nanovibration coating process in the range of up to 1.0 MHz frequency, with variety of amplitudes.

16. The apparatus according to claim 3, whereas the same frequency ranges as in claim above can be achieved, by combining vibrations of different harmonics (1^{st}, 2^{nd}, 3^{rd}, 4^{th}) of one type of vibrations (longitudinal, bending, torsion, their combination or other type).

17. A method of preventing biofilm formation associated with indwelling devices; comprising ability to form nanovibration coating process, whereas every material point of the surface is moving and there is no point, which is not moving at least in one plane surface.

18. The method of claim 17, comprising capability to excite nanovibration coating process and adjusted to elastic characteristics of the device material.

19. The method of claim 17, for nano vibration coating process, which is achieved by the combination of more than one harmonic modes of longitudinal vibration type and enables to avoid the “dead points” (inevitable while using one vibration mode).

20. The method of claim 17, comprising ability to avoid “dead points” by applying two different longitudinal vibration modes, so as not coincide, and at no time will the vibrations be zero (by amplitude, frequency, plane).

21. The method of claim 17, comprising nano vibration coating process on external and internal surface which generates transverse vibrations energy in the perpendicular directions to the wall of the device.

22. An apparatus for preventing biofilm formation associated with indwelling devices, comprising ability to form nanovibration coating process and have no “dead points”, while every material point of the surface is vibrating at least in one plane surface with the amplitude scale from several to 10.0 nanometers.

23. The apparatus according claim 22, comprising ability to form nanovibration coating process, while frequency spectrum of vibrations is in the range from several Hz to 10.0 MHz.

24. The apparatus according to claim 7 for standard indwelling medical device, whereas piezo ceramic element is connected to the medical device externally to the body.

25. The apparatus of claim 7, comprising a piezo element attached to the catheter in a position selected from the group consisting of on the side, surrounding or inside of the medical device.

26. The apparatus of claim 7, comprising at least one piezo element coated with a conducting material, enabling better energy communication with external or internal surface of the medical device.

27. The apparatus according to claim 26, wherein mechanical vibration device may have at least one piezo material body, which may have cylindrical shape and his internal, external and torsion surfaces are covered by electrodes.

28. The apparatus according to claim 27, wherein the electrodes may be divided with non-conductive places, which may be parallel or non-parallel to polarization direction; and the single phase, two-phase, or multi phase electrical signal may be sent from controller to electrodes; and by means of different connections between electrodes longitudinal, bending and torsion vibrations may be excited simultaneously or separately.

29. The apparatus according to claim 28, whereas piezo ceramic element has a shape selected from the group consisting of ring shaped and disk shaped.

30. The apparatus of claim 2, whereas nanovibration coating effect can be reached using bending, torsion an thickness vibration modes separately or together and the effect extends to a certain distance from the piezo element in both directions of it's longitudinal axis.

31. The apparatus of claim 30, while nano vibration coating process is achieved by bending vibration type; the combination of more than one harmonic modes enables to avoid the “dead points” and at no time will the vibrations be zero (by amplitude, frequency, plane); and “dead points” of two different bending vibration modes not coincide.

32. The apparatus of claim 30, while nano vibration coating process is achieved by torsion vibration type; the combination of more than one harmonic mode enables to avoid the “dead points” and at no time will the vibrations be zero (by amplitude, frequency, plane); and “dead points” of two different torsion vibration modes not coincide.

33. The apparatus of claim 30, comprising the electrodes on the surfaces of cylindrical piezo element divided into different shapes (two or more electrodes).

34. The method of claim 17, comprising ability to actuate various combinations of vibration modes simultaneously and changed periodically; and all vibration modes may be achieved on one element.

35. The method of claim 34, whereas the above effect may be achieved by using separate sections of piezo element, which together form cylindrical shape (or other hollow shape) and each of them must have multiple electrode sections on the surface.

36. The method of claim 35, whereas the above effect can be achieved by placement of multiple piezo elements around the device, each having only one electrode.

37. The method of claim 1, comprising nano vibration coating process which can be directed and focused at a determinate part of standard medical device: in particular it can be directed to act either on part of device outside the body, or at the determinate part of device inside the body.

38. The method of claim 1, whereas piezo element enables in addition to nano vibration coating process to achieve the effect of pushing or pulling materials on said surfaces, including fluids and particulates suspended in them.

39. The method of claim 38, while specific combinations of longitudinal and bending vibration modes (1st harmonic of longitudinal and 2nd harmonic of bending) are used to actuate the piezo element.

40. The method of claim 38, comprising piezo element's ability to manipulate with waves front with backward and forward acceleration; the direction of the movements can be simultaneously opposite one to another on each opposing surfaces.

41. The method of claim 1, comprising ability to form nanovibration coating process, whereas this process is achieved with cylindrical piezo element and may form standing waves in the liquid (which is in contact with this cylindrical piezo element) and considerable micro pressure changes occur, resulting in partial or whole dissinfection and killing bacteria in the liquid.

42. The apparatus of claim 41, while the cylindrical piezo element may be of different shapes, having rotation axis and to excite the process the bending and torsion vibration modes must be applied simultaneously.

43. The apparatus of claim 42, comprising the cylinidrical piezo element, whereas the standing wave may constitute barrier and block the ability of bacteria to enter and whereas pulsing standing waves can contribute to the effect, to expel out biological matter.

44. The apparatus of claim 2, whereas piezo element may be attached to standard medical device in a manner, when external surface of vibration device is attached to internal surface of medical device.

45. The apparatus of claim 2, whereas piezo element may be attached to standard hollow medical device, such as catheter, in a manner, when internal surface of vibration device is attached to external surface of medical device.

46. The apparatus of claim 2, whereas piezo element may be attached to standard medical device in a manner, when one piece of vibration device is used and it's external and internal surfaces attached to two different, hollow medical devices.

47. The apparatus of claim 2, whereas piezo element may be attached to standard medical device which has thick wall and two pieces of vibration device can be applied—one internally and another externally.

48. The apparatus of claim 2, comprising lengthy medical devices, which may require multi piezo elements to achieve the desired effect and when the medical device is furnished with actuators having two or more characteristic signals; and the following is achieved: the length of walls of the device is vibrated in natural vibration of longitudinal, bending and torsion modes simultaneously.

49. The apparatus of claim 48, whereas the said effect can be achieved by either attaching the actuators internally or externally to medical device surface.

50. The apparatus of claim 48, whereas at least two shapes of the group consisting of convex, concave and tapered can be used for piezo element shape.

51. The apparatus of claim 2, whereas piezo element can be directly attached to the medical device, or by use of standard or specifically designed connectors (one or more, having different physical mechanical properties).

52. A method of preventing biofilm formation associated with indwelling devices, comprising ability to form nanovibration coating process, while this process can be controlled directionally through all length of the device, by intensity and time, and this ability influences on the reduction of biofilm formation.

53. A method for preventing biofilm formation associated with indwelling devices, comprising ability to form nanovibration coating process, whereas the process may be excited in the portion of the device or overall it's length.

54. A method of preventing biofilm formation associated with indwelling devices, comprising ability to form nanovibration coating process, whereas feedback—sensing function is possible, for the purpose of adjustment.

55. A method of preventing biofilm formation associated with indwelling devices, comprising ability to form nanovibration coating process, which enables to expel biological matter (body secretions normally blocked by foreign devices) out of the body and as a result to decline the biofilm formation process.

56. The method of claim 1, whereas transverse vibration energy effects the fluids in contact and the friction of the fluids is reduced, the vibration may expel the fluid and drying process at the point of contact with the skin occur, which effect in resistant to the bacteria entry.

57. The method of claim 56, which slows or prevents the entry of bacteria at the point between the skin and external wall of the device, at the point of device entry, into the body.

58. The method of claim 56, comprising transversal energy, which effects the surrounding tissues and prevents the establishment of biofilms.

59. The method of claim 1, avoiding at the point and the whole part of the device which entry into vascular system (vein, artery, etc.) thrombus attachment and grows.

60. The method of claim 1, whereas the frequent thrombus and the attachment of the matter on the tip face is prevented and effects in reduce friction of the liquid, flowing throw the device, when the liquid is pushed or pulled of the body, regardless of the direction, and prevents the attachment of any particular matter.

61. The method of claim 60, comprising nano vibration coating process, which reduce dynamic friction of the liquid in the contact with the medical device, improving the flow and speeding up drying, when needed.

62. The method of claim 1, comprising ability to form nanovibration coating process, whereas the energy of this process may have a transverse character, that means the energy may be transferred to the tissues of the human body, from external surface.

63. A method of preventing biofilm formation associated with indwelling devices, comprising ability to form nanovibration coating process, which reduces friction and mechanical stress during the introduce and withdraw of the medical device.

64. A method of preventing biofilm formation associated with indwelling medical devices comprising: an ability utilization of different vibration energies to create different conditions and encourage to grow separate bacteria and to preference the other, in other words—to select the bacteria (as bacteria differ in their ability to attach and form communities).

65. The method of claim 1, comprising one or more catheters from the group consisting of an IV catheter, urinary catheter, a gastric catheter, a lung catheter, and cardiovascular catheter.

66. The apparatus of claim 2 for achieving nanovibration coating in standard peripheral IV catheter, consisting of standard medical IV catheter and at least one piezo element, attached to the connector or to the hub of the said device.

67. The apparatus of claim 2, comprising one piezo element, which can be used as sensor, and the other as a piezo element for nano vibration coating process and these piezo elements are excited from controller, which both controls and receivers signals from sensor.

68. The apparatus of claim 2, locating the electrical signal controller and source of energy by attaching on the rest of the hand and allowing free movement of the hand.

69. The apparatus of claim 2, comprising piezo element, which can be placed on any part of the line including starting from the fluid bag, pumps or any ancillary equipment connected to the system; one or more piezo elements can be used on each of the points, which can serve as entry point for microorganisms.

70. The apparatus of claim 2, comprising piezo element, which can be attached to adhesive aid band (plaster) and by this way attached to standard-medical device.

71. The apparatus of claim 2, providing nano vibration coating process in central vascular catheters/or urinary catheter, applicable in single and multiple channels, whereas the piezo element can be placed on the convergence of the channels or on each of them separately.

72. The apparatus of claim 71, comprising nano vibration coating process in urinary catheter, in which the piezo element can be placed on the connector, on the part of the catheter which is outside of the urinary tract, on the urinary bag separately or on all of them together for the purpose of biofilm and incrustation prevention.

73. The apparatus of claim 2, comprising nano vibration coating process, for endothrahial ventilation tube, which are major cause of death due to pneumonia, (resulting from biofilms formation).

74. The apparatus of claim 2 comprising nano vibration coating process for the ventilation machine which becomes contaminated in standard practice and enable to prevention of biofilm formation at any part of the system, which can be furnished with piezo elements.

75. The apparatus of claim 2 comprising nano vibration coating process whereas the body tissues which are in contact with activated medical device are protected; arterial, venous, cavities, organs, mucosal membranes are protected from the colonization of bacteria and formation of biofilms.

76. The method of claim 1 comprising nano vibration coating process which can be incorporated or embedded or integrated other wise attached to completely new designed medical devices and accessories.

77. A method for nanovibration coating process all over surfaces of indwelling medical device; a method comprising ability to stimulate or release nitric oxide from targeted organs, or tissue, or small area of it.

78. The apparatus for nanovibration coating process all over surfaces of indwelling medical device, comprising ability to stimulate or release nitric oxide from targeted organs, or tissue, or small area of it.

79. A medical apparatus, comprising:
an indwelling medical device capable of being coated with a biofilm;
at least one means for generating nanovibrations, the nanovibrations traveling along surfaces of the device;
a processor to supply at least one electric signal to initiate operation of the means for generating nanovibrations.

80. The apparatus according to claim 79 wherein the nanovibrations have a frequency ranging from about 10 KHz to about 100 MHz.

81. The apparatus according to claim 80 wherein the nanovibrations have a frequency ranging from about 4 MHz to about 80 MHz.

82. The apparatus according to claim 79 wherein the nanovibrations have amplitudes ranging from about 0.001 to about 100 nanometers.

83. The apparatus according to claim 79 wherein the nanovibrations have amplitudes ranging from about 0.1 to about 50 nanometers.

84. The apparatus according to claim 79 wherein the means for generating vibrations generates at least two nanovibrations of different energies which energies have different inhibitory effects upon different types of bacteria.

85. The apparatus according to claim 79 wherein the means for generating nanovibrations comprises at least two piezo ceramic bodies.

86. The apparatus according to claim 85 wherein one of the at least two piezo ceramic bodies generates strongest nanovibrations on an internal surface of the medical device and a second of the at least two piezo ceramic bodies generates strongest nanovibrations on an external surface of the medical device.

87. The apparatus according to claim 79 wherein the means for generating nanovibrations generates the nanovibrations transverse to a longitudinal length of the medical device.

88. The apparatus according to claim 79 wherein the means for generating nanovibrations ensures elimination of dead points where amplitude and frequency are zero.

89. The apparatus according to claim 79 wherein the medical device is a catheter.

90. The apparatus according to claim 79 further comprising a device for receiving information on the status of the nanovibration travel and an information display selected from the group consisting of an audio signal, a video signal and combinations thereof.

91. The apparatus according to claim 79 wherein nanovibrations are restricted to travel along surfaces of the medical device but not through walls of the medical device.

92. A method for inhibiting microorganism growth on medical devices comprising:
connecting to a medical device a means for generating nanovibrations; and
transmitting electrical signals to the means for generating nanovibrations from a signal computer processing unit;
wherein the generated nanovibrations inhibit the formation of microorganisms on surfaces of the medical device.

93. A method according to claim 92 wherein the nanovibrations have a frequency ranging from about 10 KHz to about 100 MHz.

94. A method according to claim 92 wherein the nanovibrations have an amplitude ranging from about 0.001 to about 100 nanometers.

95. A method according to claim 92 wherein the nanovibrations are generated to promulgate transverse to a longitudinal length of the medical device.
